(19) United States
(12) Patent Application Publication
(10) Pub. No.: US 2024/0148755 A1
(43) Pub. Date: May 9, 2024

(54) WATER-BASED PHARMACEUTICAL COMPOSITION CONTAINING URSODEOXYCHOLIC ACID OR SALT THEREOF

(71) Applicant: SANTEN PHARMACEUTICAL CO., LTD., Osaka-shi, Osaka (JP)

(72) Inventors: Yoko ENDO, Ikoma-shi,Nara (JP); Yusuke OTOMARU, lkoma-shi, Nara (JP); Hitoshi SASAKI, lkoma-shi, Nara (JP); Tatsuya HATA, lkoma-shi, Nara (JP); Tomoko ODA, lkoma-shi, Nara (JP)

(73) Assignee: SANTEN PHARMACEUTICALCO., LTD., Osaka-shi, Osaka (JP)

(21) Appl. No.: 18/546,135

(22) PCT Filed: Feb. 14, 2022
(86) PCT No.: PCT/JP2022/005718

§ 371 Date: Aug. 11, 2023

(30) Foreign Application Priority Data
Feb. 15, 2021 (JP) 2021-022001

(51) Int. Cl. A61K 31/575; A61K 47/18; A61K 47/26; A61P 27/10

(52) U.S. Cl. CPC A61K 31/575; A61K 47/18; A61K 47/186; A61K 47/26; A61P 27/10

(57) ABSTRACT

The present disclosure provides a pharmaceutical composition comprising ursodeoxycholic acid or a salt thereof, a preservative, and water.

### TECHNICAL FIELD

The present invention relates to an aqueous pharmaceutical composition comprising ursodeoxycholic acid or a salt thereof.

### BACKGROUND ART

Ursodeoxycholic acid is a compound that promotes bile secretion and inhibits suppresses the production of cytokines and chemokines, and is therefore used in the treatment of liver diseases (Non-Patent Document 1). Ursodeoxycholic acid is also expected to be a therapeutic or preventive medicine for presbyopia because it improves the lens elasticity (Patent Document 1). Furthermore, there is a known composition in which ursodeoxycholic acid is water-solubilized by adding an aqueous soluble starch conversion product, which may be administered orally or the like (Patent Document 2).

Tissue penetration of an active ingredient is an important factor for the active ingredient to exert its efficacy. It is desired to develop aqueous pharmaceutical preparations containing ursodeoxycholic acid or a salt thereof as an active ingredient with an improved tissue penetration of the active ingredient.

### PRIOR ART DOCUMENT

### Patent Document

Patent Document 1: WO 2020/129964
Patent Document 2: JP 2019-532082 A

### Non-Patent Document

Non-Patent Document 1: URSO^{(registered trade mark)} Tablets 50 mg URSO^{(registered trade mark)} Tablets 100 mg Package insert

### SUMMARY

### Technical Problem

An objective of the present invention is to provide an aqueous pharmaceutical composition comprising ursodeoxycholic acid or a salt thereof with improved tissue penetration characteristics of ursodeoxycholic acid.

### Solution to Problem

As a result of intensive research to solve the aforementioned problem, the present inventors have surprisingly found that a pharmaceutical composition comprising a preservative in addition to ursodeoxycholic acid or a salt thereof and water exhibits excellent tissue penetration characteristics of ursodeoxycholic acid and thereby have reached the present invention. The kinds of additives, their contents, dosages, etc. that can be used in pharmaceutical formulations are severely restricted. Within such restrictions, it is surprising and a great advantage in developing pharmaceutical formulations to find that the tissue penetration characteristics of ursodeoxycholic acid can be improved by using a preservative which are normally used as an additive, without the addition of special ingredients.

In addition, during a development of an aqueous solution composition comprising ursodeoxycholic acid, the present inventors have found that addition of a nonionic surfactant unexpectedly improves the solution stability of the composition. In particular, when a cationic preservative was used as a preservative, the inventors faced the problem that it was difficult to obtain the composition exhibiting good solubility. During investigating this problem to solve, the inventors have found that in a ursodeoxycholic acid-comprising aqueous composition, selecting benzalkonium halide as a cationic preservative and adding a nonionic surfactant can unexpectedly make it possible to exhibit a good solution stability.

Furthermore, the inventors have found that the addition of a buffer to a pharmaceutical composition which comprises ursodeoxycholic acid or a salt thereof, a preservative, and water surprisingly improves the preservative efficacy of the pharmaceutical composition. The inventors also have found that the addition of ursodeoxycholic acid or a salt thereof to a pharmaceutical composition comprising a nonionic surfactant and water suppresses the appearance change of the pharmaceutical composition.

Specifically, the invention includes the following aspects.

### [Item 1]

A pharmaceutical composition comprising ursodeoxycholic acid or a salt thereof, a preservative, and water.

### [Item 2]

The pharmaceutical composition according to Item 1, wherein the preservative is selected from benzalkonium halide, boric acid or a salt thereof, and combinations thereof.

### [Item 3]

The pharmaceutical composition according to Item 1 or 2, wherein the preservative comprises benzalkonium halide, and optionally comprises boric acid or a salt thereof.

### [Item 4]

The pharmaceutical composition according to any one of Items 1 to 3, wherein the preservative comprises benzalkonium halide and boric acid or a salt thereof.

### [Item 5]

The pharmaceutical composition according to any one of Items 2 to 4, wherein the benzalkonium halide is selected from benzalkonium chloride, benzalkonium bromide, and a combination thereof.

### [Item 6]

The pharmaceutical composition according to any one of Items 2 to 5, wherein the benzalkonium halide is benzalkonium chloride.

### [Item 7]

The pharmaceutical composition according to any one of Items 2 to 6, wherein the boric acid or a salt thereof is selected from boric acid, borax, and a combination thereof.

### [Item 8]

The pharmaceutical composition according to any one of Items 1 to 7, wherein the pharmaceutical composition has a pH greater than or equal to 8.0.

### [Item 9]

The pharmaceutical composition according to any one of Items 1 to 8, wherein the pharmaceutical composition has a pH of 8.3 to 9.3.

### [Item 10]

The pharmaceutical composition according to any one of Items 1 to 9, further comprising a nonionic surfactant.

### [Item 11]

The pharmaceutical composition according to Item 10, wherein the nonionic surfactant is polyoxyethylene sorbitan fatty acid ester.

### [Item 12]

The pharmaceutical composition according to Item 11, wherein the polyoxyethylene sorbitan fatty acid ester is polysorbate 80.

### [Item 13]

The pharmaceutical composition according to any one of Items 1 to 12, further comprising a buffer.

### [Item 14]

The pharmaceutical composition according to Item 13, wherein the buffer is selected from phosphoric acid or a salt thereof, citric acid or a salt thereof, acetic acid or a salt thereof, carbonic acid or a salt thereof, tartaric acid or a salt thereof, ε-aminocaproic acid or a salt thereof, trometamol or a salt thereof, and combinations thereof.

### [Item 15]

The pharmaceutical composition according to Item 13, wherein the buffer is trometamol or a salt thereof.

### [Item 16]

The pharmaceutical composition according to any one of Items 1 to 15, wherein the content of the ursodeoxycholic acid or a salt thereof in the pharmaceutical composition is 0.00001 to 5% (w/v).

### [Item 17]

The pharmaceutical composition according to any one of Items 1 to 16, wherein the content of the ursodeoxycholic acid or a salt thereof in the pharmaceutical composition is 0.0003 to 0.9% (w/v).

### [Item 18]

The pharmaceutical composition according to any one of Items 1 to 17, wherein the content of the preservative in the pharmaceutical composition is 0.0001 to 3.5% (w/v).

### [Item 19]

The pharmaceutical composition according to any one of Items 2 to 18, wherein the content of the benzalkonium halide in the pharmaceutical composition is 0.0001 to 0.05% (w/v).

### [Item 20]

The pharmaceutical composition according to any one of Items 2 to 19, wherein the content of the boric acid or a salt thereof in the pharmaceutical composition is 0.001 to 3% (w/v).

### [Item 21]

The pharmaceutical composition according to any one of Items 10 to 20, wherein the content of the nonionic surfactant in the pharmaceutical composition is 0.001 to 30 parts by mass relative to 1 part by mass of ursodeoxycholic acid or a salt thereof.

### [Item 22]

The pharmaceutical composition according to any one of Items 10 to 21, wherein the content of the nonionic surfactant in the pharmaceutical composition is 0.001 to 0.3% (w/v).

### [Item 23]

The pharmaceutical composition according to any one of Items 10 to 22, wherein the content of the nonionic surfactant in the pharmaceutical composition is 0.03 to 0.09% (w/v).

### [Item 24]

The pharmaceutical composition according to any one of Items 13 to 23, wherein the content of the buffer in the pharmaceutical composition is 0.001 to 5% (w/v).

### [Item 25]

The pharmaceutical composition according to any one of Items 14 to 24, wherein the content of the trometamol or a salt thereof in the pharmaceutical composition is 0.001 to 2% (w/v).

### [Item 26]

The pharmaceutical composition according to any one of Items 14 to 25, wherein the content of the trometamol or a salt thereof in the pharmaceutical composition is 0.05 to 0.9% (w/v).

### [Item 27]

The pharmaceutical composition according to any one of Items 1 to 26, further comprising glycerin.

### [Item 28]

The pharmaceutical composition according to any one of Items 1 to 27, which is a solution.

### [Item 29]

The pharmaceutical composition according to any one of Items 1 to 28, wherein the pharmaceutical composition is administered into eye.

### [Item 30]

The pharmaceutical composition according to any one of Items 1 to 29, wherein the pharmaceutical composition is an eye drop.

### [Item 31]

The pharmaceutical composition according to any one of Items 1 to 30, wherein the pharmaceutical composition is for treating and/or preventing presbyopia, an eye disease accompanied by a decrease in lens elasticity, or an eye disease accompanied by a decrease in accommodative function of the eye.

### [Item 32]

Use of the pharmaceutical composition according to any one of Items 1 to 30, in the manufacture of a medicament for treating and/or preventing presbyopia, an eye disease accompanied by a decrease in lens elasticity, or an eye disease accompanied by a decrease in accommodative function of the eye.

### [Item 33]

A method for treating and/or preventing presbyopia, an eye disease accompanied by a decrease in lens elasticity, or an eye disease accompanied by a decrease in accommodative function of the eye, comprising administering to a subject in need thereof a therapeutically and/or prophylactically effective amount of the pharmaceutical composition according to any one of Items 1 to 30.

### [Item 34]

A method for improving tissue penetration of ursodeoxycholic acid or a salt thereof in a pharmaceutical composition comprising ursodeoxycholic acid or a salt thereof and water, comprising adding a preservative.

### [Item 35]

A method for improving solution stability of a pharmaceutical composition comprising ursodeoxycholic acid or a salt thereof, a preservative, and water, comprising adding a nonionic surfactant.

### [Item 36]

A method for suppressing an appearance change of a pharmaceutical composition comprising a nonionic surfactant and water, comprising adding ursodeoxycholic acid or a salt thereof.

### [Item 37]

A method for suppressing an appearance change of a pharmaceutical composition comprising a nonionic surfactant, a preservative, and water, comprising adding ursodeoxycholic acid or a salt thereof.

### [Item 38]

The method according to Item 35 or 37, wherein the preservative comprises benzalkonium halide.

### [Item 39]

A method for improving preservative efficacy of a pharmaceutical composition comprising ursodeoxycholic acid or a salt thereof, a preservative, and water, comprising adding a buffer.

Each of the elements described in the above Items 1 to 39 may be optionally selected and combined.

### Advantageous Effect of Invention

The present invention provides an aqueous pharmaceutical composition having an excellent tissue penetration characteristics of ursodeoxycholic acid or a salt thereof.

### DESCRIPTION OF EMBODIMENTS

Embodiments of the present invention are described in detail below.

In one aspect, the present disclosure provides a pharmaceutical composition comprising ursodeoxycholic acid (hereinafter sometimes referred to as “UDCA”) or a salt thereof (hereinafter sometimes referred to as “the active ingredient of the present invention”), a preservative, and water (hereinafter sometimes referred to as “the pharmaceutical composition of the present disclosure”). The pharmaceutical composition of the present disclosure may exhibit an excellent tissue penetration of the active ingredient of the present invention.

In one aspect, this disclosure provides a method for improving tissue penetration of ursodeoxycholic acid or a salt thereof in a pharmaceutical composition comprising ursodeoxycholic acid or a salt thereof and water, comprising adding a preservative.

The pharmaceutical composition of the present disclosure may be used for treating and/or preventing presbyopia, an eye disease accompanied by a decrease in lens elasticity, or an eye disease accompanied by a decrease in accommodative function of the eye.

In one aspect, this disclosure provides a pharmaceutical composition comprising ursodeoxycholic acid or a salt thereof, a preservative, and water, and further comprising a nonionic surfactant. Said pharmaceutical composition may have an excellent tissue penetration characteristics of the active ingredient of the present invention and may have an improved solution stability.

In one aspect, this disclosure provides a method for improving solution stability of a pharmaceutical composition comprising ursodeoxycholic acid or a salt thereof, a preservative, and water, comprising adding a nonionic surfactant.

In one aspect, this disclosure provides a pharmaceutical composition comprising ursodeoxycholic acid or a salt thereof, a preservative, and water, and further comprising a buffer. Said pharmaceutical composition may have an excellent tissue penetration characteristics of the active ingredient of the present invention and may have an improved preservative efficacy.

In one aspect, this disclosure provides a method for improving preservative efficacy of a pharmaceutical composition comprising ursodeoxycholic acid or a salt thereof, a preservative, and water, comprising adding a buffer.

In one aspect, this disclosure provides a pharmaceutical composition comprising ursodeoxycholic acid or a salt thereof, a preservative, and water, and further comprising a nonionic surfactant and a buffer. Said pharmaceutical composition may have an excellent tissue penetration characteristics of the active ingredient of the present invention, may have an improved solution stability, and may have an improved preservative efficacy.

In one aspect, this disclosure provides a method for improving solution stability and a preservative efficacy of a pharmaceutical composition comprising ursodeoxycholic acid or a salt thereof, a preservative, and water, comprising adding a nonionic surfactant and a buffer. In one embodiment, the preservative comprises benzalkonium halide.

In one embodiment, in the pharmaceutical composition of the present disclosure, the preservative comprises benzalkonium halide, boric acid or a salt thereof, or combinations thereof.

In one embodiment, in the pharmaceutical composition of the present disclosure, the preservative comprises benzalkonium halide.

In one embodiment, in the pharmaceutical composition of the present disclosure, the nonionic surfactant comprises polyoxyethylene sorbitan fatty acid ester.

In one embodiment, in the pharmaceutical composition of the present disclosure, the preservative comprises benzalkonium halide, the nonionic surfactant comprises polyoxyethylene sorbitan fatty acid ester.

In this disclosure, “ursodeoxycholic acid” or “UDCA” is a compound represented by the following formula:

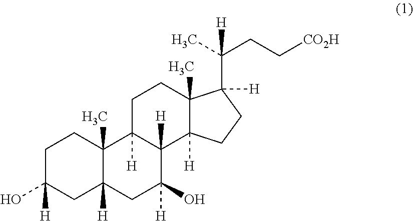

(CAS Registration Number:128-13-2), also called ursodiol and 3α,7β-Dihydroxy-5β-cholan-24-oic acid.

In this disclosure, ursodeoxycholic acid may be in a salt form, the salt form is not particularly limited as long as it is a pharmaceutically acceptable salt. Examples of such a salt include inorganic salts such as hydrochlorides, hydrobromides, hydroiodides, nitrates, sulfates, phosphates, etc.; organic acid salts such as acetates, trifluoroacetates, benzoates, oxalates, malonates, succinates, maleates, fumarates, tartrates, citrates, methanesulfonates, ethanesulfonates, trifluoromethanesulfonates, benzenesulfonates, p-toluenesulfonates, glutamates, aspartates, etc.; metal salts such as sodium salts, potassium salts, calcium salts, magnesium salts, etc.; inorganic salts such as ammonium salts, etc.; and organic amine salts such as triethylamine salts, guanidine salts, etc. Preferred examples include sodium salts and potassium salts.

Ursodeoxycholic acid or a salt thereof used for the pharmaceutical composition of the present disclosure may be in the form of hydrates or solvates.

In this disclosure, ursodeoxycholic acid or a salt thereof may be prepared according to usual methods in the field of organic chemistry or may be obtained commercially.

The content of ursodeoxycholic acid or a salt thereof in the pharmaceutical composition of the present disclosure is not specifically limited. The lower limit of the content is preferably 0.00001% (w/v), more preferably 0.0001% (w/v), still preferably 0.0003% (w/v), still more preferably 0.001% (w/v), particularly preferably 0.003% (w/v), particularly more preferably 0.01% (w/v), particularly more preferably 0.03% (w/v), most preferably 0.07% (w/v). The upper limit of the content is preferably 5% (w/v), more preferably 3% (w/v), still more preferably 2% (w/v), still more preferably 1% (w/v), particularly preferably 0.9% (w/v), particularly more preferably 0.7% (w/v), particularly more preferably 0.5% (w/v), most preferably 0.35% (w/v). Especially, from a viewpoint of preservative efficacy of the pharmaceutical composition, the upper limit of the content of ursodeoxycholic acid or a salt thereof is preferably 1% (w/v), more preferably 0.9% (w/v), still more preferably 0.7% (w/v), particularly preferably 0.5% (w/v), most preferably 0.35% (w/v). A preferably range of the content of ursodeoxycholic acid or a salt thereof can be indicated by a combination of the lower and upper limits exemplified above, and is preferably 0.00001 to 5% (w/v), more preferably 0.0001 to 3% (w/v), still more preferably 0.0003 to 2% (w/v), still more preferably 0.001 to 1% (w/v), particularly preferably 0.003 to 0.9% (w/v), particularly more preferably 0.01 to 0.7% (w/v), particularly more preferably 0.03 to 0.5% (w/v), most preferably 0.07 to 0.35% (w/v). Especially, from a viewpoint of preservative efficacy of the pharmaceutical composition, the content of ursodeoxycholic acid or a salt thereof preferably 0.0001 to 1% (w/v), more preferably 0.0003 to 0.9% (w/v), still more preferably 0.001 to 0.7% (w/v), particularly preferably 0.003 to 0.5% (w/v), particularly more preferably 0.01 to 0.35% (w/v), particularly more preferably 0.03 to 0.35% (w/v), most preferably 0.07 to 0.35% (w/v). The content of ursodeoxycholic acid or a salt thereof may also be 0.1 to 0.3% (w/v). From a viewpoint of solution stability of the pharmaceutical composition, the content of ursodeoxycholic acid or a salt thereof is preferably 0.01% (w/v) or more, for example, 0.03% (w/v) or more.

In the present disclosure, the term “% (w/v)” means a mass (g) of a target ingredient comprised in 100 mL of a pharmaceutical composition. For example, “0.01% (w/v) of ursodeoxycholic acid” means that the amount of ursodeoxycholic acid comprised in 100 mL of a pharmaceutical composition is 0.01 g.

In this disclosure, when a salt of ursodeoxycholic acid is used to formulate a pharmaceutical composition, the content of ursodeoxycholic acid or a salt thereof in the pharmaceutical composition may base on the mass of the salt added into the pharmaceutical composition or may base on the mass converted as ursodeoxycholic acid, preferably base on the mass converted as ursodeoxycholic acid. In the present invention, when ursodeoxycholic acid or a salt thereof, being in the form of hydrates or solvates, is used to formulate a pharmaceutical composition, the content of ursodeoxycholic acid or a salt thereof may base on the mass of hydrates or solvates of ursodeoxycholic acid or a salt thereof, or may base on the mass converted as ursodeoxycholic acid or a salt thereof, preferably base on the mass converted as ursodeoxycholic acid.

The same applies below unless otherwise stated.

A preservative used in the pharmaceutical composition of the present disclosure are not particularly limited as long as it can be used as an additive for a pharmaceutical product. A single kind of preservative may be used, or any combinations of two or more kinds of preservative may be used.

Examples of such a preservative used in the pharmaceutical composition of the present disclosure include:
cationic preservatives such as benzalkonium halide (benzalkonium chloride, benzalkonium bromide, etc.), benzethonium halide (benzethonium chloride, benzethonium bromide, etc.), chlorhexidine, chlorhexidine gluconate, polyquaternium-1, polyhexamethylene biguanide;
anionic preservatives such as boric acid or a salt thereof, sorbic acid, potassium sorbate, methyl parahydroxybenzoate, propyl parahydroxybenzoate;
neutral preservatives such as chlorobutanol.

From a viewpoint of a particularly excellent tissue penetration of ursodeoxycholic acid or a salt thereof, benzalkonium chloride and/or benzalkonium bromide are particularly preferred, and benzalkonium chloride most preferred.

In one embodiment of the present invention, from a viewpoint of tissue penetration, the preservative comprised in the pharmaceutical composition of the present disclosure is selected from benzalkonium halide, boric acid or a salt thereof, and combinations thereof. In this embodiment, in order to ensure preservative efficacy of the pharmaceutical composition of the present disclosure, the pharmaceutical composition of the present disclosure may comprise other ingredient(s) that may function as a preservative in addition to benzalkonium halide and/or boric acid or a salt thereof. Alternatively, the pharmaceutical composition of the present disclosure may not comprise other ingredient(s) that may function as a preservative in an amount that can exert preservative efficacy.

In one embodiment of the present invention, from a viewpoint of tissue penetration, the preservative comprised in the pharmaceutical composition of the present disclosure comprises benzalkonium halide, and optionally comprises boric acid or a salt thereof. In this embodiment, in order to ensure preservative efficacy of the pharmaceutical composition of the present disclosure, the pharmaceutical composition of the present disclosure may comprise other ingredient(s) that may function as a preservative in addition to benzalkonium halide and/or boric acid or a salt thereof. Alternatively, the pharmaceutical composition of the present disclosure may not comprise other ingredient(s) that may function as a preservative in an amount that can exert preservative efficacy.

In one embodiment of the present invention, from a viewpoint of tissue penetration and preservative efficacy, the preservative comprised in the pharmaceutical composition of the present disclosure comprises benzalkonium halide and boric acid or a salt thereof. In this embodiment, in order to ensure preservative efficacy of the pharmaceutical composition of the present disclosure, the pharmaceutical composition of the present disclosure may comprise other ingredient(s) that may function as a preservative in addition to benzalkonium halide and boric acid or a salt thereof. Alternatively, the pharmaceutical composition of the present disclosure may not comprise other ingredient(s) that may function as a preservative in an amount that can exert preservative efficacy.

In this disclosure, examples of the term “benzalkonium halide” include benzalkonium chloride and benzalkonium bromide. A preferred example of “benzalkonium halide” is benzalkonium chloride.

In this disclosure, examples of “boric acid or a salt thereof” include alkali metal salts of boric acid such as potassium tetraborate, sodium borate, potassium borate, borax, potassium metaborate, alkaline earth metal salts of boric acid (calcium salts, magnesium salts), hydrates of borates, and combinations of boric acid and borates. Preferred examples of “boric acid or a salt thereof” include boric acid, borax, and combinations thereof.

In this disclosure, the content of the preservative in the pharmaceutical composition is not particularly limited. The lower limit of the content is preferably 0.0001% (w/v), more preferably 0.001% (w/v), still more preferably 0.003% (w/v), still more preferably 0.004% (w/v), particularly preferably 0.005% (w/v), particularly more preferably 0.006% (w/v), particularly more preferably 0.00675% (w/v), most preferably 0.0075% (w/v). The upper limit of the content is preferably 3.5% (w/v), more preferably 2% (w/v), still more preferably 1.5% (w/v), still more preferably 1.2% (w/v), particularly preferably 1% (w/v), particularly more preferably 0.8% (w/v), particularly more preferably 0.6% (w/v), most preferably 0.5% (w/v).

A preferred range of the content of the preservative may be indicated by a combination of the upper and lower limits exemplified above, and is preferably 0.0001 to 3.5% (w/v), more preferably 0.001 to 2% (w/v), still more preferably 0.003 to 1.5% (w/v), still more preferably 0.004 to 1.2% (w/v), particularly preferably 0.005 to 1% (w/v), particularly more preferably 0.006 to 0.8% (w/v), particularly more preferably 0.00675 to 0.6% (w/v), most preferably 0.0075 to 0.5% (w/v).

In a case where the pharmaceutical composition of the present disclosure comprises two or more kinds of preservative, the content of the preservative may indicate the total content of these two or more kinds of preservative.

In this disclosure, in a case where benzalkonium halide is comprised in the pharmaceutical composition, its content is not particularly limited. The lower limit of the content is preferably 0.0001% (w/v), more preferably 0.001% (w/v), still more preferably 0.003% (w/v), still more preferably 0.004% (w/v), particularly preferably 0.005% (w/v), particularly more preferably 0.006% (w/v), particularly more preferably 0.00675% (w/v), most preferably 0.0075% (w/v). The upper limit of the content is preferably 0.05% (w/v), more preferably 0.04% (w/v), still more preferably 0.035% (w/v), still more preferably 0.03% (w/v), particularly preferably 0.025% (w/v), particularly more preferably 0.02% (w/v), particularly more preferably 0.015% (w/v), most preferably 0.01% (w/v).

Especially, from a viewpoint of preservative efficacy of the pharmaceutical composition, the lower limit of the content of benzalkonium halide is preferably 0.005% (w/v), more preferably 0.006% (w/v), still more preferably 0.00675% (w/v), most preferably 0.0075% (w/v).

A preferred range of the content of benzalkonium halide may be indicated by a combination of the upper and lower limits exemplified above, and is preferably 0.0001 to 0.05% (w/v), more preferably 0.001 to 0.04% (w/v), still more preferably 0.003 to 0.035% (w/v), still more preferably 0.004 to 0.03% (w/v), particularly preferably 0.005 to 0.025% (w/v), particularly more preferably 0.006 to 0.02% (w/v), particularly more preferably 0.00675 to 0.015% (w/v), most preferably 0.0075 to 0.01% (w/v).

In particular, from a viewpoint of preservative efficacy of the pharmaceutical composition, the content of benzalkonium halide is preferably 0.005 to 0.025% (w/v), more preferably 0.006 to 0.02% (w/v), still more preferably 0.00675 to 0.015% (w/v), most preferably 0.0075 to 0.01% (w/v).

In addition, the lower limit of the content of benzalkonium halide in the pharmaceutical composition of the present disclosure is, relative to 1 part by mass of ursodeoxycholic acid or a salt thereof, preferably 0.001 parts by mass, more preferably 0.002 parts by mass, still more preferably 0.005 parts by mass, still more preferably 0.01 parts by mass, particularly preferably 0.02 parts by mass, most preferably 0.03 parts by mass. The upper limit of the content of benzalkonium halide in the pharmaceutical composition of the present disclosure is, relative to 1 part by mass of ursodeoxycholic acid or a salt thereof, for example, 1 parts by mass, preferably 0.5 parts by mass, more preferably 0.2 parts by mass, still more preferably 0.1 parts by mass, still more preferably 0.09 parts by mass, particularly preferably 0.08 parts by mass, most preferably 0.075 parts by mass. A preferred range of the content of benzalkonium halide may be indicated by a combination of the upper and lower limits exemplified above and is, relative to 1 part by mass of ursodeoxycholic acid or a salt thereof, for example, 0.001 to 1 parts by mass, preferably 0.001 to 0.5 parts by mass, more preferably 0.002 to 0.2 parts by mass, still more preferably 0.005 to 0.1 parts by mass, still more preferably 0.01 to 0.09 parts by mass, particularly preferably 0.02 to 0.08 parts by mass, most preferably 0.03 to 0.075 parts by mass.

In a case where the pharmaceutical composition of the present disclosure comprises two or more kinds of benzalkonium halide, the content of benzalkonium halide indicates the total content of these two or more kinds of benzalkonium halide.

In this disclosure, in a case where boric acid or a salt thereof is comprised in the pharmaceutical composition, its content is not particularly limited. The lower limit of the content is preferably 0.001% (w/v), more preferably 0.005% (w/v), still more preferably 0.01% (w/v), still more preferably 0.05% (w/v), particularly preferably 0.1% (w/v), particularly more preferably 0.2% (w/v), particularly more preferably 0.25% (w/v), most preferably 0.3% (w/v). The upper limit of the content is preferably 3% (w/v), more preferably 1.5% (w/v), still more preferably 1.2% (w/v), still more preferably 1% (w/v), particularly preferably 0.8% (w/v), particularly more preferably 0.5% (w/v), particularly more preferably 0.4% (w/v), most preferably 0.35% (w/v). In particular, from a viewpoint of preservative efficacy of the pharmaceutical composition, the lower limit of the content of boric acid or a salt thereof is preferably 0.05% (w/v), more preferably 0.1% (w/v), still more preferably 0.15% (w/v), particularly preferably 0.2% (w/v), particularly more preferably 0.25% (w/v), most preferably 0.3% (w/v), and the upper limit of the content of boric acid or a salt thereof is preferably 1.2% (w/v), more preferably 1% (w/v), still more preferably 0.8% (w/v), particularly preferably 0.5% (w/v), particularly more preferably 0.4% (w/v), most preferably 0.35% (w/v). A preferred range of the content of boric acid or a salt thereof may be indicated by a combination of the upper and lower limits exemplified above, and is preferably 0.001 to 3% (w/v), more preferably 0.005 to 1.5% (w/v), still more preferably 0.01 to 1.2% (w/v), still more preferably 0.05 to 1% (w/v), particularly preferably 0.1 to 0.8% (w/v), particularly more preferably 0.2 to 0.5% (w/v), particularly more preferably 0.25 to 0.4% (w/v), most preferably 0.3 to 0.35% (w/v). In particular, from a viewpoint of preservative efficacy of the pharmaceutical composition, the content of boric acid or a salt thereof is preferably 0.05 to 1.2% (w/v), more preferably 0.1 to 1% (w/v), still more preferably 0.15 to 0.8% (w/v), particularly preferably 0.2 to 0.5% (w/v), particularly more preferably 0.25 to 0.4% (w/v), most preferably 0.3 to 0.35% (w/v).

In a case where the pharmaceutical composition of the present disclosure comprises two or more kinds of boric acid or a salt thereof, the content of boric acid or a salt thereof indicates the total content of these two or more kinds of boric acid or a salt thereof. Boric acid or a salt thereof may also function as buffers, for example, but the exemplary contents of boric acid or a salt thereof descried above comprise the amount of boric acid or a salt thereof comprised in the pharmaceutical compositions of this disclosure for purposes other than such preservatives. That is, the content of boric acid or a salt thereof described above indicates the total content of boric acid or a salt thereof comprised in the pharmaceutical composition of this disclosure, regardless of their use or purpose.

In this disclosure, a pH of the pharmaceutical composition is not particularly limited, but is preferably alkaline in a case where ursodeoxycholic acid or a salt thereof are to be dissolved. The pH of the pharmaceutical composition of the present disclosure is preferably 8.0 or more. The lower limit of pH is preferably 8.1, more preferably 8.2, still more preferably 8.3, particularly preferably 8.4, most preferably 8.5. In particular, from a viewpoint of preservative efficacy of the pharmaceutical composition, the lower limit of pH is preferably 8.3, more preferably 8.4. The upper limit of pH is preferably 10.0, more preferably 9.5, still more preferably 9.3, particularly preferably 9.1, most preferably 9.0. A preferred range of the pH may be indicated by a combination of the upper and lower limits exemplified above, and is preferably 8.0 to 10.0, more preferably 8.1 to 9.5, still more preferably 8.2 to 9.3, still more preferably 8.3 to 9.3, particularly preferably 8.4 to 9.3, particularly more preferably 8.4 to 9.1, most preferably 8.4 to 9.0.

In one embodiment of the pharmaceutical composition of the present disclosure, the pharmaceutical composition of the present disclosure further comprises a buffer. A single kind of buffer may be used, or any combinations of two or more kinds of buffer may be used.

In this disclosure, the buffer which may be comprised is not particularly limited as long as it can be used as an additive for a pharmaceutical product. Examples of such a buffer include phosphoric acid or a salt thereof, citric acid or a salt thereof, acetic acid or a salt thereof, carbonic acid or a salt thereof, tartaric acid or a salt thereof, ε-aminocaproic acid, trometamol or a salt thereof, etc. Examples of a salt of phosphoric acid include sodium phosphate, sodium dihydrogenphosphate, disodium hydrogenphosphate, potassium phosphate, potassium dihydrogen phosphate, dipotassium hydrogenphosphate, etc. Examples of a salt of citric acid include sodium citrate, disodium citrate, trisodium citrate, etc. Examples of a salt of acetic acid include sodium acetate, potassium acetate, etc. Examples of a salt of carbonic acid include sodium carbonate, sodium hydrogen carbonate, etc. Examples of a salt of tartaric acid include sodium tartrate, potassium tartrate, etc. Examples of a salt of trometamol include its hydrochloride etc. From a viewpoint of preservative efficacy of the pharmaceutical composition of the present disclosure, the buffer is preferably trometamol or a salt thereof.

In one embodiment of the present invention, from a viewpoint of preservative efficacy, a buffer comprised in the pharmaceutical composition of the present disclosure is trometamol or a salt thereof. In this embodiment, in order to ensure buffering effect of the pharmaceutical composition of the present disclosure, the pharmaceutical composition of the present disclosure may comprise other ingredient(s) that may function as a buffer in addition to trometamol and a salt thereof. Alternatively, the pharmaceutical composition of the present disclosure may not comprise other ingredient(s) that may function as a buffer in an amount that can exert buffering effect.

In this disclosure, in a case where a buffer is comprised in the pharmaceutical composition, the content of the buffer in the pharmaceutical composition is not particularly limited. The lower limit of the content is preferably 0.001% (w/v), more preferably 0.01% (w/v), still more preferably 0.05% (w/v), still more preferably 0.1% (w/v), particularly preferably 0.2% (w/v), particularly more preferably 0.4% (w/v), particularly more preferably 0.5% (w/v), most preferably 0.6% (w/v). The upper limit of the content is preferably 5% (w/v), more preferably 3% (w/v), still more preferably 2.5% (w/v), still more preferably 2% (w/v), particularly preferably 1.5% (w/v), particularly more preferably 1% (w/v), particularly more preferably 0.8% (w/v), most preferably 0.65% (w/v). In particular, from a viewpoint of preservative efficacy of the pharmaceutical composition, the lower limit of the content of the buffer is preferably 0.1% (w/v), more preferably 0.2% (w/v), still more preferably 0.4% (w/v), particularly preferably 0.5% (w/v), most preferably 0.6% (w/v), and the upper limit of the content of the buffer is preferably 2% (w/v), more preferably 1.5% (w/v), still more preferably 1% (w/v), particularly preferably 0.8% (w/v), most preferably 0.65% (w/v). A preferred range of the content of the buffer may be indicated by a combination of the upper and lower limits exemplified above, and is preferably 0.001 to 5% (w/v), more preferably 0.01 to 3% (w/v), still more preferably 0.05 to 2.5% (w/v), still more preferably 0.1 to 2% (w/v), particularly preferably 0.2 to 1.5% (w/v), particularly more preferably 0.4 to 1% (w/v), particularly more preferably 0.5 to 0.8% (w/v), most preferably 0.6 to 0.65% (w/v). In particular, from a viewpoint of preservative efficacy of the pharmaceutical composition, the content of the preservative is preferably 0.1 to 2% (w/v), more preferably 0.2 to 1.5% (w/v), still more preferably 0.4 to 1% (w/v), particularly preferably 0.5 to 0.8% (w/v), most preferably 0.6 to 0.65% (w/v). In a case where the pharmaceutical composition of the present disclosure comprises two or more kinds of buffer, the content of buffer may indicate the total content of these two or more kinds of buffer.

In this disclosure, in a case where trometamol or a salt thereof is comprised as a buffer in the pharmaceutical composition, the content of trometamol or a salt thereof is not particularly limited. The lower limit of the content is preferably 0.001% (w/v), more preferably 0.005% (w/v), still more preferably 0.01% (w/v), still more preferably 0.02% (w/v), particularly preferably 0.05% (w/v), particularly more preferably 0.1% (w/v), particularly more preferably 0.15% (w/v), most preferably 0.2% (w/v). The upper limit of the content is preferably 2% (w/v), more preferably 1.5% (w/v), still more preferably 1.2% (w/v), still more preferably 1% (w/v), particularly preferably 0.7% (w/v), particularly more preferably 0.5% (w/v), particularly more preferably 0.4% (w/v), most preferably 0.3% (w/v). In particular, from a viewpoint of preservative efficacy of the pharmaceutical composition, the lower limit of the content of trometamol or a salt thereof is preferably 0.05% (w/v), more preferably 0.1% (w/v), still more preferably 0.12% (w/v), particularly preferably 0.15% (w/v), most preferably 0.2% (w/v), and the upper limit of the content trometamol or a salt thereof is preferably 0.9% (w/v), more preferably 0.7% (w/v), still more preferably 0.5% (w/v), particularly preferably 0.4% (w/v), most preferably 0.3% (w/v). A preferred range of the content of trometamol or a salt thereof may be indicated by a combination of the upper and lower limits exemplified above, and is preferably 0.001 to 2% (w/v), more preferably 0.005 to 1.5% (w/v), still more preferably 0.01 to 1.2% (w/v), still more preferably 0.02 to 1% (w/v), particularly preferably 0.05 to 0.7% (w/v), particularly more preferably 0.1 to 0.5% (w/v), particularly more preferably 0.15 to 0.4% (w/v), most preferably 0.2 to 0.3% (w/v). In particular, from a viewpoint of preservative efficacy of the pharmaceutical composition, the content of trometamol or a salt thereof is preferably 0.05 to 0.9% (w/v), more preferably 0.1 to 0.7% (w/v), still more preferably 0.12 to 0.5% (w/v), particularly preferably 0.15 to 0.4% (w/v), most preferably 0.2 to 0.3% (w/v).

In one embodiment of the pharmaceutical composition of the present invention, the pharmaceutical composition of the present disclosure further comprises a nonionic surfactant. In the present invention, the nonionic surfactant which may be comprised is not particularly limited as long as it can be used as an additive for a pharmaceutical product. A single kind of nonionic surfactant may be used, or any combinations of two or more kinds of nonionic surfactant may be used. Examples of such a nonionic surfactant include polyoxyethylene castor oil, polyoxyethylene hydrogenated castor oil, polyoxyethylene sorbitan fatty acid ester, vitamin E TPGS, polyoxyethylene fatty acid ester, polyoxyethylene polyoxypropylene glycol, sucrose fatty acid ester etc. From a viewpoint of solution stability and preservative efficacy of the pharmaceutical composition, polyoxyethylene sorbitan fatty acid ester is preferred.

As the polyoxyethylene castor oil, for example, various polyoxyethylene castor oils with different numbers of polymerization of ethylene oxide can be used, and the number of polymerization of ethylene oxide is preferably 5 to 100, more preferably 20 to 50, particularly preferably 30 to 40, most preferably 35. Specific examples of polyoxyethylene castor oil include polyoxyl 5 castor oil, polyoxyl 9 castor oil, polyoxyl 15 castor oil, polyoxyl 35 castor oil, polyoxyl 40 castor oil, etc.

As the polyoxyethylene hydrogenated castor oil, for example, various polyoxyethylene hydrogenated castor oils with different numbers of polymerization of ethylene oxide can be used, and the number of polymerization of ethylene oxide is preferably 10 to 100, more preferably 20 to 80, particularly preferably 40 to 70, most preferably 60. Specific examples of polyoxyethylene hydrogenated castor oil include polyoxyethylene hydrogenated castor oil 10, polyoxyethylene hydrogenated castor oil 40, polyoxyethylene hydrogenated castor oil 50, polyoxyethylene hydrogenated castor oil 60, etc.

Examples of polyoxyethylene sorbitan fatty acid ester include polysorbate 80, polysorbate 65, polysorbate 60, polysorbate 40, polyoxyethylene sorbitan monolaurate, polyoxyethylene sorbitan trioleate, etc. Polysorbate 80 is most preferred.

Vitamin E TPGS is also referred to as tocopherol polyethylene glycol 1000 succinate.

Examples of polyoxyethylene fatty acid ester include polyoxyl 40 stearate, etc.

Examples of polyoxypropylene glycol include polyoxyethylene (160) polyoxypropylene (30) glycol, polyoxyethylene (42) polyoxypropylene (67) glycol, polyoxyethylene (54) polyoxypropylene (39) glycol, polyoxyethylene (196) polyoxypropylene (67) glycol, polyoxyethylene (20) polyoxypropylene (20) glycol, etc.

Examples of sucrose fatty acid ester include sucrose stearate, etc.

In this disclosure, in a case where a nonionic surfactant is comprised in the pharmaceutical composition, the content of the nonionic surfactant is not particularly limited. The lower limit of the content is preferably 0.001% (w/v), more preferably 0.005% (w/v), still more preferably 0.01% (w/v), still more preferably 0.02% (w/v), particularly preferably 0.03% (w/v), particularly more preferably 0.04% (w/v), most preferably 0.05% (w/v). The upper limit of the content is preferably 0.3% (w/v), more preferably 0.2% (w/v), still more preferably 0.15% (w/v), still more preferably 0.1% (w/v), particularly preferably 0.09% (w/v), particularly more preferably 0.08% (w/v), most preferably 0.075% (w/v). In particular, from a viewpoint of stability of the pharmaceutical composition, the lower limit of the content of the nonionic surfactant is preferably 0.03% (w/v), more preferably 0.04% (w/v), most preferably 0.05%, and from a viewpoint of preservative efficacy of the pharmaceutical composition, the upper limit of the content of the nonionic surfactant is preferably 0.09% (w/v), more preferably 0.08% (w/v), most preferably 0.075% (w/v). A preferred range of the content of the nonionic surfactant may be indicated by a combination of the upper and lower limits exemplified above, and is preferably 0.001 to 0.3% (w/v), more preferably 0.005 to 0.2% (w/v), still more preferably 0.01 to 0.15% (w/v), still more preferably 0.02 to 0.1% (w/v), particularly preferably 0.03 to 0.09% (w/v), particularly more preferably 0.04 to 0.08% (w/v), most preferably 0.05 to 0.075% (w/v). In particular, from a viewpoint of stability and preservative efficacy of the pharmaceutical composition, the content of is the nonionic surfactant preferably 0.03 to 0.09% (w/v), more preferably 0.04 to 0.08% (w/v), most preferably 0.05 to 0.075% (w/v).

In one embodiment, the lower limit of the content of the nonionic surfactant in the pharmaceutical composition of the present disclosure is, relative to 1 part by mass of ursodeoxycholic acid or a salt thereof, for example, 0.001 parts by mass, preferably 0.005 parts by mass, more preferably 0.01 parts by mass, still more preferably 0.03 parts by mass, still more preferably 0.05 parts by mass, particularly preferably 0.1 parts by mass, most preferably 0.15 parts by mass. The upper limit of the content of is a nonionic surfactant in the pharmaceutical composition of the present invention is, relative to 1 part by mass of ursodeoxycholic acid or a salt thereof, for example, 30 parts by mass, preferably 10 parts by mass, more preferably 5 parts by mass, still more preferably 2.5 parts by mass, still more preferably 0.9 parts by mass, particularly preferably 0.8 parts by mass, most preferably 0.75 parts by mass. A preferred range of the content of the nonionic surfactant may be indicated by a combination of the upper and lower limits exemplified above, and is, relative to 1 part by mass of ursodeoxycholic acid or a salt thereof, for example, 0.001 to 30 parts by mass, preferably 0.005 to 10 parts by mass, more preferably 0.01 to 5 parts by mass, still more preferably 0.03 to 2.5 parts by mass, still more preferably 0.05 to 0.9 parts by mass, particularly preferably 0.1 to 0.8 parts by mass, most preferably 0.15 to 0.75 parts by mass. In one embodiment, from a viewpoint of long-term solution stability/suppression of appearance change, the content of the nonionic surfactant in the pharmaceutical composition of the present disclosure is, relative to 1 part by mass of ursodeoxycholic acid or a salt thereof, preferably 0.05 to 5 parts by mass, more preferably 0.1 to 2.5 parts by mass, still more preferably 0.15 to 1 parts by mass, still more preferably 0.15 to 0.75 parts by mass.

In one embodiment, in a case where benzalkonium halide is comprised in the pharmaceutical composition of the present disclosure, the lower limit of the content of the nonionic surfactant in the pharmaceutical composition of the present disclosure is, relative to 1 part by mass of benzalkonium halide, for example, 0.1 parts by mass, preferably 1 part by mass, more preferably 3 parts by mass, most preferably 5 parts by mass. The upper limit of the content of the nonionic surfactant in the pharmaceutical composition of the present invention is, relative to 1 part by mass of benzalkonium halide, for example, 30 parts by mass, preferably 15 parts by mass, more preferably 13 parts by mass, most preferably parts by mass. A preferred range of the content of the nonionic surfactant may be indicated by a combination of the upper and lower limits exemplified above, and is, relative to 1 part by mass of benzalkonium halide, for example, 0.1 to 30 parts by mass, preferably 1 to 15 parts by mass, more preferably 3 to 13 parts by mass, most preferably 5 to 10 parts by mass. In a case where the pharmaceutical composition of the present disclosure comprises two or more kinds of nonionic surfactant, the content of the nonionic surfactant indicates the total content of these two or more kinds of nonionic surfactant.

In one embodiment, in the pharmaceutical composition of the present disclosure,
the content of ursodeoxycholic acid or a salt thereof is 0.05 to 0.15% (w/v) (preferably 0.07 to 0.13% (w/v)),
the content of a nonionic surfactant is 0.05 to 0.95 parts by mass (preferably 0.15 to 0.9 parts by mass) relative to 1 part by mass of ursodeoxycholic acid or a salt thereof, and,
the content of a nonionic surfactant is 3 to 16 parts by mass (preferably 6 to 14 parts by mass), relative to 1 part by mass of benzalkonium halide.

In one embodiment, in the pharmaceutical composition of the present disclosure,
the content of ursodeoxycholic acid or a salt thereof is 0.25 to 0.35% (w/v) (preferably 0.27 to 0.33% (w/v)),
the content of a nonionic surfactant is 0.05 to 0.2 parts by mass (preferably 0.15 to 0.18 parts by mass) relative to 1 part by mass of ursodeoxycholic acid or a salt thereof, and
the content of a nonionic surfactant is 3 to 7 parts by mass (preferably 4 to 6 parts by mass), relative to 1 part by mass of benzalkonium halide.

In one embodiment of the present invention, from a viewpoint of solution stability and/or preservative efficacy of the pharmaceutical composition, a nonionic surfactant comprised in the pharmaceutical composition of the present disclosure is polyoxyethylene sorbitan fatty acid ester. In this embodiment, the pharmaceutical composition of the present disclosure may comprise other nonionic surfactant(s) in addition to polyoxyethylene sorbitan fatty acid ester. Alternatively, the pharmaceutical composition of the present disclosure may not comprise other nonionic surfactant(s). The preferred content of polyoxyethylene sorbitan fatty acid ester comprised in the pharmaceutical compositions of the present disclosure is as exemplified above for a nonionic surfactant.

In one embodiment, this disclosure provides a pharmaceutical composition comprising: ursodeoxycholic acid or a salt thereof (preferably 0.00001 to 5% (w/v)),
benzalkonium chloride (preferably 0.0001 to 0.05% (w/v)), and water.

In one embodiment, this disclosure provides a pharmaceutical composition comprising:
ursodeoxycholic acid or a salt thereof (preferably 0.00001 to 5% (w/v)),
benzalkonium chloride (preferably 0.0001 to 0.05% (w/v)),
boric acid or a salt thereof (preferably 0.001 to 3% (w/v)), and
water.

In one embodiment, this disclosure provides a pharmaceutical composition comprising:
ursodeoxycholic acid or a salt thereof (preferably 0.00001 to 5% (w/v)),
benzalkonium chloride (preferably 0.0001 to 0.05% (w/v)),
boric acid or a salt thereof (preferably 0.001 to 3% (w/v)),
polyoxyethylene sorbitan fatty acid ester (preferably 0.001 to 0.3% (w/v)), and water.

In one embodiment, this disclosure provides a pharmaceutical composition comprising:
ursodeoxycholic acid or a salt thereof (preferably 0.00001 to 5% (w/v)),
benzalkonium chloride (preferably 0.0001 to 0.05% (w/v)),
boric acid or a salt thereof (preferably 0.001 to 3% (w/v)),
polyoxyethylene sorbitan fatty acid ester (preferably 0.001 to 0.3% (w/v)),
trometamol or a salt thereof (preferably 0.001 to 2% (w/v)), and
water.

Other additive(s) may optionally be used in the pharmaceutical composition of the present invention. Examples of additives include tonicity agents, stabilizers, antioxidants, high molecular weight polymers, pH adjusters, bases, etc.

The pharmaceutical composition of the present invention may optionally comprise a tonicity agent which is usable as an additive for a pharmaceutical product. Examples of such a tonicity agent include ionic tonicity agents and non-ionic tonicity agents, etc. Examples of the ionic tonicity agents include sodium chloride, potassium chloride, calcium chloride, magnesium chloride, etc.; and examples of the non-ionic tonicity agents include glycerin, propylene glycol, sorbitol, mannitol, etc. From a viewpoint of preservative efficacy of the pharmaceutical composition of the present invention, glycerin is preferred. In a case where tonicity agent(s) is used in the pharmaceutical composition of the present disclosure, the content of tonicity agent(s) may be optionally adjusted according to the type of the tonicity agent(s), and others, and is preferably 0.1 to 5% (w/v), more preferably 0.5 to 4% (w/v), still more preferably 1 to 3% (w/v), particularly preferably 1.2 to 2.5% (w/v), most preferably 1.5 to 2% (w/v).

In one embodiment, this disclosure provides a pharmaceutical composition comprising:
ursodeoxycholic acid or a salt thereof (preferably 0.00001 to 5% (w/v)),
benzalkonium chloride (preferably 0.0001 to 0.05% (w/v)),
boric acid or a salt thereof (preferably 0.001 to 3% (w/v)),
polyoxyethylene sorbitan fatty acid ester (preferably 0.001 to 0.3% (w/v)),
trometamol or a salt thereof (preferably 0.001 to 2% (w/v)),
glycerin (preferably 0.1 to 5% (w/v)) and water, and
preferably having pH 8.0 to 10.0.

The pharmaceutical composition of the present invention may optionally comprise a stabilizer which is usable as an additive for a pharmaceutical product. Examples of such a stabilizer include edetic acid, disodium edetate, etc. In a case where stabilizer(s) is used in the pharmaceutical composition of the present invention, the content of the stabilizer(s) may be optionally adjusted according to the type of the stabilizer(s), and is preferably 0.001 to 10% (w/v), more preferably 0.01 to 5% (w/v), still more preferably 0.05 to 3% (w/v), most preferably 0.1 to 2% (w/v).

The pharmaceutical composition of the present invention may optionally comprise an antioxidant which is usable as an additive for a pharmaceutical product. Examples of such an antioxidant include tocopherol, dibutylhydroxytoluene, butylhydroxyanisole, sodium erythorbate, propyl gallate, sodium sulfite, etc. In a case where antioxidant(s) is used in the pharmaceutical composition of the present invention, the content of the antioxidant(s) may be optionally adjusted according to the type of the antioxidant(s), and others, and is preferably 0.0001 to 1% (w/v), more preferably 0.0005 to 0.1% (w/v), still more preferably 0.001 to 0.02% (w/v), most preferably 0.005 to 0.010% (w/v).

The pharmaceutical composition of the present invention may optionally comprise a high molecular weight polymer which is usable as an additive for a pharmaceutical product. Examples of such a high molecular weight polymer include methylcellulose, ethylcellulose, hydroxymethylcellulose, hydroxyethylcellulose, hydroxypropylcellulose, hydroxyethylmethylcellulose, hydroxypropylmethylcellulose (hypromellose), carboxymethylcellulose, carboxymethylcellulose sodium, hydroxypropylmethylcellulose acetate succinate, hydroxypropylmethylcellulose phthalate, carboxymethylethylcellulose, cellulose acetate phthalate, polyvinylpyrrolidone, polyvinyl alcohol, carboxyvinyl polymer, polyethylene glycol, and the like. In the present invention, the high molecular weight polymer is preferably hydroxypropylmethylcellulose (hypromellose). In a case where high molecular weight polymer(s) is used in the pharmaceutical composition of the present invention, the content of the high molecular weight polymer(s) may be optionally adjusted according to the type of the high molecular weight polymer(s), and others, and is preferably in a range of 0.001 to 5% (w/v), more preferably 0.01 to 1% (w/v), and most preferably 0.1 to 0.5% (w/v).

The pharmaceutical composition of the present invention may optionally comprise a pH adjuster which is usable as an additive for a pharmaceutical product. Examples of such a pH adjuster include hydrochloric acid, sodium hydroxide, potassium hydroxide, etc. In a case where pH adjuster(s) is comprised in the pharmaceutical composition of the present disclosure, the content of the pH adjuster(s) may be optionally adjusted according to the type of the pH adjuster, and others, and is preferably 0.001 to 5% (w/v), more preferably 0.01 to 1% (w/v), more preferably 0.1 to 0.5% (w/v).

The pharmaceutical composition of the present disclosure comprises water as a base. The amount thereof is not particularly limited and can be adjusted depending on the amount of other ingredients. The grade of water is not particularly limited as long as it is pharmaceutically acceptable. Examples include purified water.

The form of the pharmaceutical composition of the present disclosure is not particularly limited as long as it is in the form of a composition comprising water as a base. Pastes, mousses, gels, solutions, emulsions, suspensions and creams can be exemplified.

In one embodiment, the form of the pharmaceutical composition of the present disclosure is a solution. In the present disclosure, “solution” refers to a liquid that is clear or transparent when observed visually.

In one embodiment, the pharmaceutical composition of the present disclosure does not comprise an aqueous soluble starch conversion product such as maltodextrin, hydroxypropyl-β-cyclodextrin, etc.

In one embodiment, the pharmaceutical composition of the present disclosure does not comprise maltodextrin.

In one embodiment, the pharmaceutical composition of the present disclosure does not comprise hydroxypropyl-β-cyclodextrin.

In one embodiment of the pharmaceutical composition of the present invention, a tonicity agent is not comprised.

In one embodiment of the pharmaceutical composition of the present invention, a stabilizer is not comprised.

In one embodiment of the pharmaceutical composition of the present invention, an antioxidant is not comprised.

In one embodiment of the pharmaceutical composition of the present invention, a high molecular weight polymer is not comprised.

Ursodeoxycholic acid or a salt thereof, which is used in the pharmaceutical composition of the present disclosure can improve lens elasticity, and is useful as a medicament for treating or preventing presbyopia.

In one embodiment, the pharmaceutical composition of the present disclosure may comprise other active ingredient(s) in addition to ursodeoxycholic acid or a salt thereof.

In one embodiment, it does not comprise active ingredient(s) other than ursodeoxycholic acid or a salt thereof because ursodeoxycholic acid or a salt thereof alone may exert sufficient pharmacological effects.

The pharmaceutical composition of the present disclosure can be administered orally or parenterally. Examples of administration route include oral administration, intravenous administration, transdermal administration, and topical ocular administration (for example, instillation, conjunctival sac administration, intravitreal administration, subconjunctival administration, sub-Tenon's capsule administration). Instillation is most preferably.

Dosage forms of the pharmaceutical composition of the present disclosure are not particularly limited as long as they can be used as pharmaceuticals, and include eye drops, eye gels, injections, etc. A particularly preferred dosage form of the pharmaceutical composition of the present invention is an eye drop. They can be produced according to usual methods in the art.

The pharmaceutical composition of the present disclosure can be stored in containers made of various materials.

In this disclosure, the term “presbyopia” means a symptom/disease that is determined to be presbyopia based on general criteria used by a physician or professional. For example, diagnostic criteria for presbyopia include: Decreased near vision is noticed as a subjective symptom in a binocular vision test, and a binocular daily life visual acuity, which is a binocular distant visual acuity measured under the same condition as daily life, is less than 0.4 at 40 cm distance (clinical presbyopia); and/or with or without subjective symptoms, under unilateral best-correction where a corrected visual acuity of one eye is equal to or more than 1.0 (decimal visual acuity), accommodative amplitude is less than 2.5 Diopters” (medical presbyopia). However, if an accommodometer etc. is not available, a simple criterion wherein a visual acuity at 40 cm is less than 0.4 may be used.

In the present disclosure, the term “an eye disease accompanied by a decrease in lens elasticity” refers to an eye disease considered in the field of ophthalmology to be accompanied by a decrease in lens elasticity, including, for example, presbyopia (e.g., presbyopia due to aging), and a hardening of the lens induced by drugs and the like.

In the present disclosure, the term “accommodation function of the eye” refers to an eye function that automatically focuses on distant and/or near objects. The term “an eye disease accompanied by a decrease in accommodative function of the eye” refers to an eye disease considered in the field of ophthalmology to be accompanied by a decrease in accommodative function of the eye, including, for example, presbyopia (e.g., presbyopia due to aging), and a hardening of the lens induced by drugs etc., and decreased accommodation function induced by seeing near objects for a long time.

In this disclosure, “subject” refers not only to humans but also to other animals, such as dogs, cats, horses, etc. The subject is preferably a mammal and more preferably a human.

In this disclosure, “treatment (treating)” and “prevention (preventing)” may include, in addition to treating and preventing a disease, alleviating symptoms of the disease, delaying progression of the disease, suppressing symptoms of the disease, and inducing improvement in symptoms of the disease.

In this disclosure, “a therapeutically and/or prophylactically effective amount of” refers to an amount can result in a therapeutic and/or preventive effect of a disease and its symptoms, or an amount that can result in a delay in the progression of a disease and its symptoms, or the like.

In this disclosure, “tissue penetration of ursodeoxycholic acid or a salt thereof” refers to penetration of ursodeoxycholic acid or a salt thereof to tissues, especially eye tissues (for example, cornea, conjunctiva, uvea, eyelid, anterior chamber, ciliary body, iris, lens, vitreous body, retina, choroid, etc.). An improved tissue penetration of ursodeoxycholic acid or a salt thereof refers to, for example, an increase in the amount of ursodeoxycholic acid or a salt thereof penetrated in tissue compared to a case where a preservative-free composition is administered. The tissue penetration of ursodeoxycholic acid or a salt thereof may be evaluated, for example, by the method according to Test Example 1 of the present application.

In this disclosure, “improving solution stability of a pharmaceutical composition” means that at least a solution of a pharmaceutical composition can be obtained and may further include that the solution state continues. Furthermore, even in a solution state, the number of particles that cannot be visually confirmed may increase in the composition, and suppression of such increase in the number of particles can also be included in “improving solution stability of a pharmaceutical composition”. Originally, ursodeoxycholic acid is a poorly water-soluble compound, and depending on the conditions, such as a preservative comprised, the pharmaceutical composition may have white turbidity/precipitation. In this disclosure, by adding a nonionic surfactant, the pharmaceutical composition may become in a solution state and the pharmaceutical composition may maintain the solution state. Especially in a case where a cationic preservative is used, white turbidity/precipitation etc. may occur in the composition, but by selecting benzalkonium halide as a cationic preservative and adding a nonionic surfactant, an aqueous solution comprising ursodeoxycholic acid may be obtained. In this case, the composition preferably does not comprise any cationic preservative other than benzalkonium halide, but other cationic preservative(s) may be comprised to the extent permitted from a viewpoint of the solution stability.

In this disclosure, “suppressing an appearance change of a pharmaceutical composition” means to suppress changes in appearance such as color of the pharmaceutical composition over time. For example, even if a pharmaceutical composition is a clear, colorless liquid at the time of preparation, it may become turbid or the like over time depending on the conditions, and suppression of such change in appearance such as turbid or the like may be included in “suppressing an appearance change of a pharmaceutical composition.” In this disclosure, by further adding ursodeoxycholic acid or a salt thereof to a pharmaceutical composition comprising a nonionic surfactant and water, the change in appearance of a pharmaceutical composition over time, especially white turbidity, may be suppressed and the initial condition at the time of preparation may be maintained. The composition may or may not comprise preservatives such as benzalkonium halide.

According to the method of improving solution stability of the pharmaceutical composition of the present disclosure, the pharmaceutical composition may maintain a dissolved state over a long period of time (for example, at room temperature, for 1 month, preferably for 3 months, more preferably for 6 months, even more preferably for 1 year, especially preferably for 2 years, most preferably for 3 years) without having white turbidity or precipitates.

In this disclosure, a method of evaluating “preservative efficacy of a pharmaceutical composition” is not particularly limited, but can be evaluated according to, for example, preservative effectiveness tests of European Pharmacopoeia (EP), Japanese Pharmacopeia (JP), United States Pharmacopeia (USP), Pharmacopoeia of the People's Republic of China (CP), and the like, and Preservative efficacy evaluation study of EXAMPLES in the present application.

In this disclosure, “improved preservative efficacy of a pharmaceutical composition” may mean improved preservative efficacy compared to, for example, a buffer-free composition.

According to the method of the present disclosure for improving preservative efficacy of a pharmaceutical composition, the pharmaceutical composition may exhibit an excellent preservative efficacy, and may conform to preservative effectiveness tests of, for example, European Pharmacopoeia (EP), Japanese Pharmacopeia (JP), United States Pharmacopeia (USP) and Pharmacopoeia of the People's Republic of China (CP).

The detailed description of the pharmaceutical composition of the present disclosure can be applied to other aspects, such as aspects of the methods disclosed herein.

### EXAMPLES

Formulation examples and results of tests are shown below for a better understanding of the present invention and are not intended to limit the scope of the present invention.

### FORMULATION EXAMPLE

Representative Formulation examples using the pharmaceutical composition of the present invention are as follows. In the following Formulation examples, the amount of each ingredient comprised in 100 mL of the composition is shown.

### Formulation Example 1

| Eye drop/Solution (in 100 mL) | pH 8.4 | |
|---|---|---|
| Ursodeoxycholic acid | 0.1 | g |
| Benzalkonium chloride | 0.0075 | g |
| Polysorbate 80 | 0.06 | g |
| Boric acid | 0.3 | g |
| Trometamol | 0.2 | g |
| Glycerin | 2.0 | g |

| | |
|---|---|
| Diluted hydrochloric acid | q.s. |
| Sodium hydroxide | q.s. |
| Purified water | q.s. |

### Formulation Example 2

| Eye drop/Solution (in 100 mL) | pH 8.5 | |
|---|---|---|
| Ursodeoxycholic acid | 0.3 | g |
| Benzalkonium chloride | 0.01 | g |
| Polysorbate 80 | 0.06 | g |
| Boric acid | 0.4 | g |
| Trometamol | 0.2 | g |
| Glycerin | 2.0 | g |

| | |
|---|---|
| Diluted hydrochloric acid | q.s. |
| Sodium hydroxide | q.s. |
| Purified water | q.s. |

### Formulation Example 3

| Eye drop/Solution (in 100 mL) | pH 8.6 | |
|---|---|---|
| Ursodeoxycholic acid | 0.3 | g |
| Benzalkonium chloride | 0.0075 | g |
| Polysorbate 80 | 0.075 | g |
| Boric acid | 0.5 | g |
| Trometamol | 0.2 | g |
| Glycerin | 1.5 | g |

| | |
|---|---|
| Diluted hydrochloric acid | q.s. |
| Sodium hydroxide | q.s. |
| Purified water | q.s. |

The above Formulation examples 1 to 3 are prepared as follows:

To a mixture of benzalkonium chloride, polysorbate 80, boric acid, trometamol, and glycerin is added purified water (80 mL), and the mixture is stirred to dissolve. Thereto is added ursodeoxycholic acid, and the mixture is stirred. Thereto is added an appropriate amount of a solution of sodium hydroxide or diluted hydrochloric acid to adjust the pH. Thereto is added an appropriate amount of purified water to a total volume of 100 mL.

### 1. Penetration Evaluation Test

Penetration of the active ingredient of the present invention into aqueous humor was evaluated.

### 1-1. Preparation of Test Formulation

Preparation of Ursodeoxycholic Acid Test Sample

To a mixture of borax 0.7 g and ursodeoxycholic acid 0.1 g was added purified water 80 mL, and the resulting mixture was stirred. Thereto was added an appropriate amount of a solution of sodium hydroxide or diluted hydrochloric acid to adjust the pH. Thereto was added an appropriate amount of purified water to a total volume of 100 mL to give a Test formulation of Example 1. The appearance of the formulation was visually observed. Test formulations of Examples 2 to 7 were also prepared in a similar way.

### 1-2. Test Method

A single dose (50 μL) of each Test formulation (Examples 1 to 7) was applied to a Japanese white rabbit (male). At 1, 2, and 4 hours after instillation, rabbit eyes were subjected to local anesthesia, and aqueous humor was collected (2 to 9 eyes per time point). Ursodeoxycholic acid concentration in the aqueous humor was measured using a high performance liquid chromatography tandem mass spectrometer (LC-MS/MS).

### 1-3. Test Results and Discussion

Test results are shown in Tables 1-1 to 1-2 below.

TABLE 1-1

| % (w/v) | Example 1 | Example 2 | Example 3 | Example 4 |
|---|---|---|---|---|
| UDCA | 0.1 | 0.1 | 0.1 | 0.1 |
| Benzalkonium | — | — | 0.01 | — |
| chloride | | | | |
| Boric acid | — | 1.4 | 0.5 | 0.5 |
| Borax | 0.7 | 1.0 | 1.0 | 1.0 |
| EDTA | — | 0.05 | — | — |
| Trometamol | — | — | 1.0 | — |
| Polysorbate 80 | — | — | 0.1 | 0.5 |
| Polyquaternium-1 | — | — | — | 0.01 |
| Glycerin | — | — | — | 0.6 |
| Purified water | q.s. | q.s. | q.s. | q.s. |
| pH | 8.5 | 8.5 | 8.5 | 8.5 |
| Appearance | Clear, | Clear, | Clear, | Clear, |
| | colorless | colorless | colorless | colorless |
| | liquid | liquid | liquid | liquid |
| Penetration to | 47.8 | 39.4 | 81.1 | 25.6 |
| aqueous humor | | | | |
| (Cmax: ng/mL) | | | | |

TABLE 1-2

| % (w/v) | Example 5 | Example 6 | Example 7 |
|---|---|---|---|
| UDCA | 0.1 | 0.1 | 0.1 |
| Benzalkonium chloride | 0.01 | 0.005 | — |
| Boric acid | 0.5 | 0.5 | 0.5 |
| Borax | 1.0 | 1.0 | 1.0 |
| Trometamol | 1.0 | 1.0 | 1.0 |
| Polysorbate 80 | 0.1 | 0.1 | 0.1 |
| Purified water | q.s. | q.s. | q.s. |
| pH | 8.5 | 8.5 | 8.5 |
| Appearance | Clear, | Clear, | Clear, |
| | colorless | colorless | colorless |
| | liquid | liquid | liquid |
| Penetration to aqueous | 72.6 | 53.1 | 42.3 |
| humor (Cmax: ng/mL) | | | |

As shown in Tables 1-1 to 1-2, Examples 1 to 7 comprising at least one a preservative (benzalkonium chloride, boric acid, borax, polyquaternium-1) exhibit excellent tissue penetration characteristics of ursodeoxycholic acid (penetration to aqueous humor). Especially, when benzalkonium chloride was comprised, better tissue penetration characteristics was confirmed.

### 2. Appearance Evaluation Test

### 2-1. Appearance Evaluation Test (1)

Appearance of compositions comprising the active ingredient of the present invention was evaluated.

### 2-1-1. Preparation of Test Formulation

Examples 8 to 15 shown in Table 2 or Table 3 were prepared in a similar way to Example 1.

### 2-1-2. Test Method

The appearance of samples immediately after preparation was visually observed. For Example 8 and Example 9 (before starting storage, after 2 weeks at 60° C., and after 1 month at 60° C.), the number of particles of 5 μm or larger was measured using a light obscuration microparticle measuring device (Beckman Coulter Life Sciences), in addition to visual observation.

### 2-1-3. Test Results and Discussion

Test results are shown in Tables 2 and 3.

TABLE 2

| % (w/v) | | Example 8 | Example 9 |
|---|---|---|---|
| UDCA | | 0.1 | 0.1 |
| Boric acid | | 0.5 | 0.5 |
| Borax | | 1.0 | 1.0 |
| EDTA | | 0.01 | 0.01 |
| Benzalkonium chloride | | 0.01 | — |
| Polysorbate 80 | | 0.1 | — |
| Trometamol | | 1.0 | 1.0 |
| Concentrated glycerin | | 0.6 | 0.6 |
| Sodium hydroxide | | q.s. | q.s. |
| Diluted hydrochloric | | q.s. | q.s. |
| acid | | | |
| Purified water | | q.s. | q.s. |
| pH | | 8.5 | 8.5 |
| Appearance | Initial | Clear, colorless | Clear, colorless |
| | | solution | solution |
| | 60° C./2 W | Clear, colorless | Clear, colorless |
| | | solution | solution |
| | 60° C./1 M | Clear, colorless | Clear, colorless |
| | | solution | solution |
| Number of | Initial | 76 | 58 |
| particulate | 60° C./2 W | 75 | 919 |
| matter of 5 μm | 60° C./1 M | 85 | 814 |
| or larger | | | |
| (particles/ | | | |
| 5 mL) | | | |

As shown in Table 2, the appearance of Example 8 and Example 9 after 1 month of storage at 60° C. was a clear, colorless solution, unchanged from the start of the test.

As for the results of particulate measurements, Example 9 comprising UDCA, boric acid, borax, EDTA, trometamol, concentrated glycerin showed an increase in the number of particulate matter. On the other hand, Example 8 further comprising benzalkonium chloride and polysorbate 80 showed no increase in the number of particulate matter.

TABLE 3

| | Example | Example | Example | Example | Example | Example |
|---|---|---|---|---|---|---|
| % (w/v) | 10 | 11 | 12 | 13 | 14 | 15 |
| UDCA | 0.1 | 0.1 | 0.1 | 0.1 | 0.1 | 0.1 |
| Benzalkonium | 0.01 | 0.01 | — | — | — | — |
| chloride | | | | | | |
| Chlorhexidine | — | — | 0.01 | — | — | — |
| gluconate | | | | | | |
| Polyquaternium-1 | — | — | — | 0.01 | — | — |
| Polyhexamethylene | — | — | — | — | 0.01 | 0.01 |
| biguanide | | | | | | |
| Polysorbate 80 | — | 0.1 | — | — | — | 1 |
| Borax | 0.7 | 0.7 | 0.7 | 0.7 | 0.7 | 0.7 |
| Sodium | 0.5 | 0.5 | 0.5 | 0.5 | 0.5 | 0.5 |
| chloride | | | | | | |
| Sodium | q.s. | q.s. | q.s. | q.s. | q.s. | q.s. |
| hydroxide | | | | | | |
| Diluted | q.s. | q.s. | q.s. | q.s. | q.s. | q.s. |
| hydrochloric | | | | | | |
| acid | | | | | | |
| Purified | q.s. | q.s. | q.s. | q.s. | q.s. | q.s. |
| water | | | | | | |
| pH | 8.5 | 8.5 | 8.5 | 8.5 | 8.5 | 8.5 |
| Appearance | White | Clear, | Precipitated | White | Precipitated | Precipitated |
| | turbidity | colorless | | turbidity | | |
| | | liquid | | | | |

As shown in Table 3, Examples 10, 12 to 14, which comprise UDCA and a cationic preservative (benzalkonium chloride, chlorhexidine gluconate, polyquaternium-1, or polyhexamethylene biguanide) but do not comprise polysorbate 80 all showed white turbidity or a generation of precipitates.

Example 11, which comprises UDCA, benzalkonium chloride, polysorbate 80, was a clear, colorless solution.

Example 15, which comprises polyhexamethylene biguanide, polysorbate 80 showed a generation of precipitates.

### 2-2-1. Appearance Evaluation Test (2)

Appearance of compositions comprising the active ingredient of the present invention was evaluated.

### 2-2-1. Preparation of Test Formulation

Example 39 to 42 and Comparative example 1 shown in Table 4 were prepared in a similar way to Example 1.

### 2-2-2. Test Method

The appearance of samples immediately after preparation was visually observed.

Appearance of each sample before starting storage, stored at 25° C. for 1 month, 3 months, 6 months, and 8 months, and stored at 40° C. for 1 month, 3 months, and 6 months was visually observed.

Evaluation Criteria are described below.
−: Clear, colorless liquid (clear, colorless)+
+: Slightly white turbid liquid (Slightly white turbid, and not clear or colorless)

### 2-2-3. Test Results and Discussion

The test results are shown in Table 4.

TABLE 4

| | | | Comparative | Example | Example | Example | Example |
|---|---|---|---|---|---|---|---|
| % (w/v) | | | example 1 | 39 | 40 | 41 | 42 |
| UDCA | | | − | 0.01 | 0.03 | 0.1 | 0.3 |
| Borax | | | 0.3 | 0.3 | 0.3 | 0.3 | 0.3 |
| Benzalkonium chloride | | | 0.01 | 0.0075 | 0.0075 | 0.0075 | 0.01 |
| Polysorbate 80 | | | 0.075 | 0.075 | 0.075 | 0.075 | 0.05 |
| Trometamol | | | 0.3 | 0.3 | 0.3 | 0.3 | 0.3 |
| Concentrated glycerin | | | 2.0 | 2.0 | 2.0 | 2.0 | 2.0 |
| Sodium hydroxide | | | q.s. | q.s. | q.s. | q.s. | q.s. |
| Diluted hydrochloric acid | | | q.s. | q.s. | q.s. | q.s. | q.s. |
| Purified water | | | q.s. | q.s. | q.s. | q.s. | q.s. |
| pH | | | 8.7 | 8.7 | 8.7 | 8.7 | 8.7 |
| Appearance | 25° C. | Initial | − | − | − | − | − |
| | | 1 M | − | − | − | − | − |
| | | 3 M | − | − | − | − | − |
| | | 6 M | − | − | − | − | − |
| | | 8 M | + | − | − | − | − |
| | 40° C. | Initial | − | − | − | − | − |
| | | 1 M | − | − | − | − | − |
| | | 3 M | + | − | − | − | − |
| | | 6 M | + | + | − | − | − |

As shown in Table 4, Comparative example 1 comprising benzalkonium chloride and polysorbate 80 but not UDCA changed to a white turbid liquid. On the other hand, Example 39 comprising UDCA in addition to benzalkonium chloride and polysorbate 80 showed a suppressed white turbidity of the liquid. Examples 40 to 42 comprising more UDCA remained a clear and colorless liquid from the start of the test.

### 3. Preservative Efficacy Evaluation Study

Preservative efficacy of compositions comprising the active ingredient of the present invention was evaluated.

### 3-1. Preparation of Test formulation

Preparation of Ursodeoxycholic Acid Test Sample

Examples 16 to 38 shown in the below table were prepared in a similar way to Example 1. Appearance of compositions was observed after preparation.

### 3-2. Test Method

(Strains)

The following strains were used as inoculum strains.
Bacteria:
   Escherichia Coli
   Pseudomonas aeruginosa
   Staphylococcus aureus
Yeast and molds:
   Candida albicans
   Aspergillus brasiliensis

(Test Procedure)

The study was conducted according to Efficacy of antimicrobial preservation defined in European Pharmacopoeia (EP). Specifically, an inoculum microorganism liquid was prepared for each strain at 10⁷ to 10⁸ cfu/mL, and axenically inoculated into each of Examples 16 to 38, followed by mixing uniformly to give a sample wherein the concentration of the inoculum strain is 10⁵ to 10⁶ cfu/mL. These samples were stored at 20 to 25° C. under a light shielding condition. At sampling points (Bacteria: 6 hours, 24 hours and 28 days after inoculation; Yeast and molds: 7 days and 28 days after inoculation), 1 mL of each sample was withdrawn with a micropipette to measure a viable count.

The viable count of bacteria, yeast and molds was measured according to Most-probable-number method defined in Microbial Limit Test of the Japanese Pharmacopoeia 17th edition.

(Method for Evaluation)

In accordance with EP's criteria (The A criteria), if a viable count met the criteria in the table below at each sampling point (Bacteria: 6 hours, 24 hours and 28 days after inoculation; Yeast and molds: 7 days and 28 days after inoculation), the test sample was evaluated as “conformed”. In addition, if all the results at each sampling point were evaluated as “conformed”, the comprehensive evaluation of preservative efficacy was evaluated as “conformed”.

hours

| | 6 hours after | 24 hours after | 7 days after | 28 days after |
|---|---|---|---|---|
| | inoculation | inoculation | inoculation | inoculation |
| Bacteria | Decrease in | Decrease in | — | No bacteria |
| | viable count | viable count | | detected |
| | by 2log or | by 3log or | | (below |
| | more from | more from | | detection |
| | initial count | initial count | | limit) |
| Yeast and | — | — | Decrease in | Equal to or |
| molds | | | viable count | less than |
| | | | by 2log or | viable count |
| | | | more from | of 7 days |
| | | | initial count | after |
| | | | | inoculation |

### 3-3. Test Results and Discussion

The test results are shown in Tables 5-1 to 5-5. Only for the results of 7 days after inoculation of Candida albicans of Examples 36 to 38, a decrease in the viable count from the initial count is represented by log reduction.

TABLE 5-1

| | | | Example | Example | Example | Example | Example | Example |
|---|---|---|---|---|---|---|---|---|
| % (w/v) | | | 16 | 17 | 18 | 19 | 20 | 21 |
| UDCA | | | 0.1 | 0.1 | 0.1 | 0.1 | 0.1 | 0.1 |
| Benzalkonium | | | 0.006 | 0.00675 | 0.006 | 0.006 | 0.01 | 0.01 |
| chloride | | | | | | | | |
| Polysorbate 80 | | | 0.075 | 0.075 | 0.075 | 0.075 | 0.1 | 0.1 |
| Boric acid | | | — | — | — | — | — | 0.5 |
| Borax | | | 0.3 | 0.3 | 0.3 | 0.3 | — | 1 |
| Trometamol | | | 0.3 | 0.3 | 0.3 | 0.3 | 1 | 1 |
| Concentrated | | | 2 | 2 | 2 | 2 | 1.5 | 0.6 |
| glycerin | | | | | | | | |
| Sodium | | | | | | | | |
| hydroxide | | | q.s. | q.s. | q.s. | q.s. | q.s. | q.s. |
| Diluted | | | | | | | | |
| hydrochloric | | | q.s. | q.s. | q.s. | q.s. | q.s. | q.s. |
| acid | | | | | | | | |
| Purified water | | | q.s. | q.s. | q.s. | q.s. | q.s. | q.s. |
| pH | | | 8.3 | 8.4 | 8.5 | 9.0 | 8.3 | 8.3 |
| Appearance | | | Clear, | Clear, | Clear, | Clear, | Clear, | Clear, |
| | | | colorless | colorless | colorless | colorless | colorless | colorless |
| | | | solution | solution | solution | solution | solution | solution |
| E. coli | 6 | hr | Conformed | Conformed | Conformed | Conformed | Conformed | Conformed |
| | 24 | hr | Conformed | Conformed | Conformed | Conformed | Conformed | Conformed |
| | 28 | day | Conformed | Conformed | Conformed | Conformed | Conformed | Conformed |
| P. | 6 | hr | Conformed | Conformed | Conformed | Conformed | Conformed | Conformed |
| aeruginosa | 24 | hr | Conformed | Conformed | Conformed | Conformed | Conformed | Conformed |
| | 28 | day | Not | Conformed | Conformed | Conformed | Conformed | Conformed |
| | | | conformed | | | | | |
| S. | 6 | hr | Conformed | Conformed | Conformed | Conformed | Conformed | Not |
| aureus | | | | | | | | conformed |
| | 24 | hr | Conformed | Conformed | Conformed | Conformed | Conformed | Conformed |
| | 28 | day | Conformed | Conformed | Conformed | Conformed | Conformed | Conformed |
| C. | 7 | day | Conformed | Conformed | Conformed | Conformed | Conformed | Conformed |
| arbicans | 28 | day | Conformed | Conformed | Conformed | Conformed | Conformed | Conformed |
| A. | 7 | day | Conformed | Conformed | Conformed | Conformed | Not | Conformed |
| brasiliensis | | | | | | | conformed | |
| | 28 | day | Conformed | Conformed | Conformed | Conformed | Conformed | Conformed |

| | | | | | | |
|---|---|---|---|---|---|---|
| Comprehensive | Not | Conformed | Conformed | Conformed | Not | Not |
| evaluation | conformed | | | | conformed | conformed |

TABLE 5-2

| | | | Example | Example | Example |
|---|---|---|---|---|---|
| % (w/v) | | | 22 | 23 | 24 |
| UDCA | | | 0.3 | 0.3 | 0.3 |
| Benzalkonium chloride | | | 0.01 | 0.01 | 0.01 |
| Polysorbate 80 | | | 0.05 | 0.05 | 0.05 |
| Borax | | | 0.3 | 0.3 | 0.3 |
| Trometamol | | | 0.3 | 0.3 | 0.3 |
| Concentrated glycerin | | | 2 | 2 | 2 |
| Sodium hydroxide | | | q.s. | q.s. | q.s. |
| Diluted hydrochloric acid | | | q.s. | q.s. | q.s. |
| Purified water | | | q.s. | q.s. | q.s. |
| pH | | | 8.3 | 8.5 | 9.0 |
| Appearance | | | Clear, | Clear, | Clear, |
| | | | colorless | colorless | colorless |
| | | | solution | solution | solution |
| E. coli | 6 | hr | Conformed | Conformed | Conformed |
| | 24 | hr | Conformed | Conformed | Conformed |
| | 28 | day | NA | Conformed | Conformed |
| P. aeruginosa | 6 | hr | Conformed | Conformed | Conformed |
| | 24 | hr | Conformed | Conformed | Conformed |
| | 28 | day | NA | Conformed | Conformed |
| S. aureus | 6 | hr | Conformed | Conformed | Conformed |
| | 24 | hr | Conformed | Conformed | Conformed |
| | 28 | day | NA | Conformed | Conformed |
| C. arbicans | 7 | day | Not | Conformed | Conformed |
| | | | conformed | | |
| | 28 | day | NA | Conformed | Conformed |
| A. brasiliensis | 7 | day | Conformed | Conformed | Conformed |
| | 28 | day | NA | Conformed | Conformed |

| Comprehensive evaluation | Not | Conformed | Conformed |
|---|---|---|---|
| | conformed | | |

NA: not assayed

As shown in Tables 5-1 and 5-2, Examples 16 to 24 showed preservative efficacy against at least one of bacteria, yeast or mold, in particular, Examples 17 to 19 and 23 to 24 showed a sufficient preservative efficacy for practical use.

The results of Tables 5-1 and 5-2 suggested that the compositions having pH8.4, pH8.5 and pH9.0 were more effective in preservative efficacy compared to the composition having pH8.3.

A comparison of the results of Example 20 and Example 21 suggested that the combined use of boric acid/borax and benzalkonium chloride was more effective in preservative efficacy against yeast and molds compared to the use of benzalkonium chloride alone as a preservative.

TABLE 5-3

| | | | Example | Example | Example | Example | Example |
|---|---|---|---|---|---|---|---|
| % (w/v) | | | 25 | 26 | 27 | 28 | 29 |
| UDCA | | | 0.1 | 0.1 | 0.3 | 0.3 | 1 |
| Benzalkonium chloride | | | 0.0075 | 0.01 | 0.01 | 0.01 | 0.01 |
| Polysorbate 80 | | | 0.075 | 0.075 | 0.05 | 0.075 | 0.05 |
| Borax | | | 0.3 | 0.3 | 0.3 | 0.3 | 0.3 |
| Trometamol | | | 0.3 | 0.3 | 0.3 | 0.3 | 0.3 |
| Concentrated glycerin | | | 2 | 2 | 2 | 2 | 2 |
| Sodium hydroxide | | | q.s. | q.s. | q.s. | q.s. | q.s. |
| Diluted hydrochloric | | | q.s. | q.s. | q.s. | q.s. | q.s. |
| acid | | | | | | | |
| Purified water | | | q.s. | q.s. | q.s. | q.s. | q.s. |
| pH | | | 8.5 | 8.5 | 8.5 | 8.5 | 8.5 |
| E. coli | 6 | hr | Conformed | Conformed | Conformed | Not | Not |
| | | | | | | conformed | conformed |
| | 24 | hr | Conformed | Conformed | Conformed | Conformed | Not |
| | | | | | | | conformed |
| | 28 | day | Conformed | Conformed | Conformed | Conformed | NA |
| P. | 6 | hr | Conformed | Conformed | Conformed | Conformed | Not |
| aeruginosa | | | | | | | conformed |
| | 24 | hr | Conformed | Conformed | Conformed | Not | Not |
| | | | | | | conformed | conformed |
| | 28 | day | Conformed | Conformed | Conformed | Not | NA |
| | | | | | | conformed | |
| S. aureus | 6 | hr | Conformed | Conformed | Conformed | Conformed | Not |
| | | | | | | | conformed |
| | 24 | hr | Conformed | Conformed | Conformed | Conformed | Not |
| | | | | | | | conformed |
| | 28 | day | Conformed | Conformed | Conformed | Conformed | NA |
| C. arbicans | 7 | day | Conformed | Conformed | Conformed | Not | Not |
| | | | | | | conformed | conformed |
| | 28 | day | Conformed | Conformed | Conformed | Conformed | NA |
| A. | 7 | day | Conformed | Conformed | Conformed | Conformed | Conformed |
| brasiliensis | 28 | day | Conformed | Conformed | Conformed | Conformed | NA |

| | | | | | |
|---|---|---|---|---|---|
| Comprehensive | Conformed | Conformed | Conformed | Not | Not |
| evaluation | | | | conformed | conformed |

As shown in Table 5-3, Examples 25 to 29 showed preservative efficacy against at least one of bacteria, yeast or mold. In particular, Examples 25 to 27 showed a sufficient preservative efficacy for practical use.

TABLE 5-4

| | | | Example | Example | Example | Example | Example | Example |
|---|---|---|---|---|---|---|---|---|
| % (w/v) | | | 30 | 31 | 32 | 33 | 34 | 35 |
| UDCA | | | 0.1 | 0.1 | 0.1 | 0.1 | 0.1 | 0.3 |
| Benzalkonium | | | 0.006 | 0.00675 | 0.00675 | 0.00675 | 0.006 | 0.01 |
| chloride | | | | | | | | |
| Polysorbate 80 | | | 0.05 | 0.075 | 0.08 | 0.09 | 0.1 | 0.05 |
| Borax | | | 0.3 | 0.3 | 0.3 | 0.3 | 0.3 | 0.3 |
| Trometamol | | | 0.3 | 0.3 | 0.3 | 0.3 | 0.3 | 0.3 |
| Concentrated | | | 2 | 2 | 2 | 2 | 2 | 2 |
| glycerin | | | | | | | | |
| Sodium hydroxide | | | q.s. | q.s. | q.s. | q.s. | q.s. | q.s. |
| Diluted | | | q.s. | q.s. | q.s. | q.s. | q.s. | q.s. |
| hydrochloric acid | | | | | | | | |
| Purified water | | | q.s. | q.s. | q.s. | q.s. | q.s. | q.s. |
| pH | | | 8.5 | 8.5 | 8.5 | 8.5 | 8.5 | 8.5 |
| E. coli | 6 | hr | Conformed | Conformed | Conformed | Conformed | Conformed | Conformed |
| | 24 | hr | Conformed | Conformed | Conformed | Conformed | Conformed | Conformed |
| | 28 | day | Conformed | Conformed | Conformed | Conformed | Conformed | Conformed |
| P. | 6 | hr | Conformed | Conformed | Conformed | Conformed | Conformed | Conformed |
| aeruginosa | 24 | hr | Conformed | Conformed | Conformed | Conformed | Conformed | Conformed |
| | 28 | day | Conformed | Conformed | Conformed | Conformed | Conformed | Conformed |
| S. aureus | 6 | hr | Conformed | Conformed | Conformed | Conformed | Not | Conformed |
| | | | | | | | conformed | |
| | 24 | hr | Conformed | Conformed | Conformed | Conformed | Conformed | Conformed |
| | 28 | day | Conformed | Conformed | Conformed | Conformed | Conformed | Conformed |
| C. | 7 | day | Conformed | Conformed | Conformed | Conformed | Conformed | Conformed |
| arbicans | 28 | day | Conformed | Conformed | Conformed | Conformed | NA | Conformed |
| A. | 7 | day | Conformed | Conformed | Conformed | Conformed | Conformed | Conformed |
| brasiliensis | 28 | day | Conformed | Conformed | Conformed | Conformed | NA | Conformed |

| | | | | | | |
|---|---|---|---|---|---|---|
| Comprehensive | Conformed | Conformed | Conformed | Conformed | Not | Conformed |
| evaluation | | | | | conformed | |

As shown in Table 5-4, Examples 30 to 35 showed preservative efficacy against at least one of bacteria, yeast or mold. In particular, Examples 30 to 33 and 35 showed a sufficient preservative efficacy for practical use.

TABLE 5-5

| | | | Example | Example | Example |
|---|---|---|---|---|---|
| % (w/v) | | | 36 | 37 | 38 |
| UDCA | | | 0.1 | 0.1 | 0.1 |
| Benzalkonium | | | 0.01 | 0.01 | 0.01 |
| chloride | | | | | |
| Polysorbate 80 | | | 0.075 | 0.075 | 0.075 |
| Borax | | | 0.3 | 0.3 | 0.3 |
| Trometamol | | | — | 0.05 | 0.3 |
| Concentrated | | | 2 | 2 | 2 |
| glycerin | | | | | |
| Sodium hydroxide | | | q.s. | q.s. | q.s. |
| Diluted | | | | | |
| hydrochloric acid | | | q.s. | q.s. | q.s. |
| Purified water | | | q.s. | q.s. | q.s. |
| pH | | | 8.5 | 8.5 | 8.5 |
| E. coli | 6 | hr | Conformed | Conformed | Conformed |
| | 24 | hr | Conformed | Conformed | Conformed |
| | 28 | day | Conformed | Conformed | Conformed |
| P. | 6 | hr | Conformed | Conformed | Conformed |
| aeruginosa | 24 | hr | Conformed | Conformed | Conformed |
| | 28 | day | Conformed | Conformed | Conformed |
| S. aureus | 6 | hr | Conformed | Conformed | Conformed |
| | 24 | hr | Conformed | Conformed | Conformed |
| | 28 | day | Conformed | Conformed | Conformed |
| C. | 7 | day | 2.5 | 4.3 | 4.3 |
| arbicans | | | (Conformed) | (Conformed) | (Conformed) |
| | 28 | day | Conformed | Conformed | Conformed |
| A. | 7 | day | Conformed | Conformed | Conformed |
| brasiliensis | 28 | day | Conformed | Conformed | Conformed |

| | | | |
|---|---|---|---|
| Comprehensive | Conformed | Conformed | Conformed |
| evaluation | | | |

As shown in Table 5-5, Examples 36 to 38 showed a potent preservative efficacy. However, the preservative efficacy against C. arbicans of Example 36, which does not comprise trometamol was weak compared with Example 37 and Example 38, both of which comprise trometamol. This result suggests that tromethamol contributes to the enhancement of preservative efficacy.

### 4. Evaluation of Drug Efficacy (1)

The effects of suspension compositions comprising UDCA on the lens elasticity were examined. The tests were conducted with reference to the methods described in Invest Ophthalmol Vis Sci, 57, 2851-2863, 2016, which evaluated the effects of EV06 on the lens elasticity.

EV06 is lipoic acid choline ester (also known as UNR 844), and has been disclosed to be useful for the treating of presbyopia. Eye drops comprising lipoic acid choline ester are in clinical development in the United States. EV06 is a compound represented by the following formula:

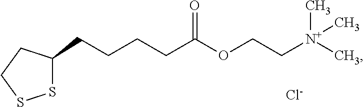 [SMILES: C[N+](C)(C)CCOC(=O)CCCC[C@@H]1CCSS1 ; [Cl-]]

which was used to compare to ursodeoxycholic acid.

### 4-1. Preparation of Test Formulation

### 1) Preparation of Vehicle

A vehicle (pH6.7) comprising 0.1% (w/v) of ethyl pyruvate, 0.269% (w/v) of sodium dihydrogenphosphate monohydrate (NaH₂PO₄H₂O), 0.433% (w/v) of disodium hydrogenphosphate (Na₂HPO₄), 0.2% (w/v) of hydroxypropylmethylcellulose, 0.5% (w/v) of NaCl, and purified water (appropriate amount) was prepared.

2) Preparation of Ursodeoxycholic Acid Test Sample

Ursodeoxycholic acid was sonicated with the addition of the vehicle to prepare a 3.0% (w/v) suspension (pH6.7). The resulting 3.0% (w/v) suspension was diluted with the vehicle to prepare a 1.0% (w/v) suspension (pH6.7). Further, the resulting 1.0% (w/v) suspension was diluted with the vehicle to prepare a 0.3% (w/v) suspension (pH6.7). The total amount of each sample to be used in one day was prepared before use.

### 3) Preparation of EV06 Sample

EV06 was sonicated with the addition of the vehicle to prepare a 1.5% (w/v) solution. The total amount of the sample to be used in one day was prepared before use.

### 4-2. Test Method

1) Each test sample (2.5 μL/eye) was instilled into the right eye of 8-month-old C57BL/6J mice with a Pipetman once per day (QD; around 9:00), twice per day (BID; around 9:00 and 17:00), or 3 times per day (TID; around 9:00, 13:00 and 17:00) for 14 days.
2) After the final instillation, the mice were euthanized by carbon dioxide inhalation, and then the eyeballs were extracted and rinsed with Hank's balanced salt solution (HBSS).
3) The sclera near the optic nerve was cut with a razor, the lens was removed through the incision, and the removed lens was immersed in HBSS.
4) The lens was placed on a glass slide, and an all-in-one fluorescence microscope BZ-9000 (Keyence) was used to capture an image of the lens (Image a).
5) Next, one cover glass (Corning^{(registered trade mark)} 22×22 mm Square) was placed on the lens, and an image in which the thickness of the lens changed due to the weight was similarly captured (Image b).
6) A change in the lens diameter was calculated from Equation 1 wherein the lens diameter of Image a is subtracted from the lens diameter of Image b, as described below. Then, the lens elasticity improvement of each sample group compared with the vehicle control group was calculated from Equation 2 described below. The mean of the vehicle control group was based on 5 eyes, the mean of each ursodeoxycholic acid sample group was based on 10 eyes, and the mean of each EV06 sample group was based on 10 eyes.

Change in lens diameter=Lens diameter in Image b of each test sample−Lens diameter in Image a of each test sample (Equation 1)

Lens elasticity improvement of each sample group=Mean change in lens diameter of each Test sample group−Mean change in lens diameter of Vehicle control group (Equation 2)

### 4-3. Test Results and Discussion

The results are shown in Table 6.

TABLE 6

| | Lens elasticity |
|---|---|
| | improvement (μm) |
| 0.3% ursodeoxycholic acid sample (QD) | 2.8 |
| 1% ursodeoxycholic acid sample (QD) | 28.1 |
| 3% ursodeoxycholic acid sample (QD) | 30.4 |
| 1.5% EV06 sample (QD) | −3.6 |
| 1.5% EV06 sample (BID) | 15.7 |
| 1.5% EV06 sample (TID) | 29.5 |

As shown in Table 6, the suspension compositions comprising 1% or more of UDCA showed a potent lens elasticity improvement.

### 5. Evaluation of Drug Efficacy (2)

The effects of solution compositions comprising UDCA on the lens elasticity were examined.

### 5-1. Preparation of Test Formulation

### 1) Preparation of Vehicle

### Vehicle-A

Vehicle-A (pH8.5) comprising 0.3% (w/v) of borax, 0.0075% (w/v) of benzalkonium chloride, 0.075% (w/v) of polysorbate 80, 0.3% (w/v) of trometamol, 2.0% (w/v) of concentrated glycerin, sodium hydroxide (appropriate amount), diluted hydrochloric acid (appropriate amount) and purified water (appropriate amount) was prepared.

### Vehicle-B

Vehicle-B (pH6.7) comprising 0.1% (w/v) of ethyl pyruvate, 0.269% (w/v) of sodium dihydrogenphosphate monohydrate (NaH₂PO₄·H₂O), 0.433% (w/v) of disodium hydrogenphosphate (Na₂HPO₄), 0.2% (w/v) of hydroxypropylmethylcellulose, 0.5% (w/v) of NaCl, and purified water (appropriate amount) was prepared.

### 2) Preparation of Ursodeoxycholic Acid Solution

To a mixture of borax (0.3% (w/v)), benzalkonium chloride (0.0075% (w/v)), polysorbate 80 (0.075% (w/v)), trometamol (0.3% (w/v)), concentrated glycerin (2.0% (w/v)) was added ursodeoxycholic acid, and the resulting mixture was stirred. The pH of the mixture was adjusted by adding sodium hydroxide (appropriate amount), diluted hydrochloric acid (appropriate amount). Thereto was added purified water (appropriate amount) to fill up to the final volume to give 0.003% (w/v), 0.01% (w/v), 0.03% (w/v), or 0.1% (w/v) ursodeoxycholic acid solutions (pH8.5).

To a mixture of borax (0.3% (w/v)), benzalkonium chloride (0.01% (w/v)), polysorbate 80 (0.05% (w/v)), trometamol (0.3% (w/v)), and concentrated glycerin (2.0% (w/v)) was added ursodeoxycholic acid, and the resulting mixture was stirred. The pH of the mixture was adjusted by adding sodium hydroxide (appropriate amount), diluted hydrochloric acid (appropriate amount). Thereto was added purified water (appropriate amount) to fill up to the final volume to give a 0.3% (w/v) ursodeoxycholic acid solution (pH8.5).

### 3) Preparation of EV06 Sample

EV06 was sonicated with the addition of Vehicle-B to prepare a 1.5% (w/v) solution. The total amount of the sample to be used in one day was prepared before use.

### 5-2. Test Method

1) Each test sample (2.5 μL/eye) was instilled into the right eye of 8-month-old C57BL/6J mice with a Pipetman once per day (QD; around 9:00), twice per day (BID; around 9:00 and 17:00), or 3 times per day (TID; around 9:00, 13:00 and 17:00) for 14 days.
2) After the final instillation, the mice were euthanized by carbon dioxide inhalation, and then the eyeballs were extracted and rinsed with Hank's balanced salt solution (HBSS).
3) The sclera near the optic nerve was cut with a razor, the lens was removed through the incision, and the removed lens was immersed in HBSS.
4) The lens was placed on a glass slide, and an all-in-one fluorescence microscope BZ-9000 (Keyence) was used to capture an image of the lens (Image a).
5) Next, one cover glass (Corning (registered trade mark) 22×22 mm Square) was placed on the lens, and an image in which the thickness of the lens changed due to the weight was similarly captured (Image b).
6) A change in the lens diameter was calculated from Equation 1 wherein the lens diameter of Image a is subtracted from the lens diameter of Image b, as described below. Then, the lens elasticity improvement of each sample group compared with the vehicle-A control group was calculated from Equation 2 described below. The mean of the vehicle-A control group was based on 5 eyes, the mean of each ursodeoxycholic acid sample group was based on 10 eyes, and the mean of each EV06 sample group was based on 10 eyes.

Change in lens diameter=Lens diameter in Image b of each test sample−Lens diameter in Image a of each test sample (Equation 1)

Lens elasticity improvement of each sample group=Mean change in lens diameter of each Test sample group−Mean change in lens diameter of Vehicle-A control group (Equation 2)

### 5-3. Test Results and Discussion

The results are shown in Table 7.

TABLE 7

| | Lens elasticity |
|---|---|
| | improvement (μm) |
| 0.003% UDCA solution (QD) | 15.6 |
| 0.01% UDCA solution (QD) | 21.5 |
| 0.03% UDCA solution (QD) | 40.8 |
| 0.1% UDCA solution (QD) | 52.3 |
| 0.3% UDCA solution (QD) | 60.4 |
| 1.5% EV06 sample (BID) | 13.1 |
| 1.5% EV06 sample (TID) | 27.4 |

As shown in Table 7, the solution compositions comprising 0.01% or more of UDCA showed a lens elasticity improvement, and the solution compositions comprising 0.03% or more of UDCA showed a potent lens elasticity improvement. It was revealed that a solution composition comprising UDCA showed a lens elasticity improvement effect from a lower concentration of UDCA compared to a suspension pharmaceutical composition comprising UDCA.

### 6. Evaluation of Drug Efficacy (3)

The effect of a solution of comprising an aqueous soluble starch conversion product and UDCA on the lens elasticity was examined.

### 6-1. Preparation of Test Formulation

### 1) Preparation of Vehicle

### Vehicle-C

Vehicle-C (pH7.0) comprising 0.1% (w/v) of ethyl pyruvate, 0.27% (w/v) of sodium dihydrogenphosphate monohydrate (NaH₂PO₄·H₂O), 0.43% (w/v) of disodium hydrogenphosphate (Na₂HPO₄), 0.2% (w/v) of hydroxypropylmethylcellulose, 0.5% (w/v) of NaCl, 5% (w/v) of hydroxypropyl-β-cyclodextrin, sodium hydroxide (appropriate amount), diluted hydrochloric acid (appropriate amount) and purified water (appropriate amount) was prepared.

### Vehicle-D

Vehicle-D (pH7.0) comprising 0.1% (w/v) of ethyl pyruvate, 0.27% (w/v) of sodium dihydrogenphosphate monohydrate (NaH₂PO₄·H₂O), 0.43% (w/v) of disodium hydrogenphosphate (Na₂HPO₄), 0.2% (w/v) of hydroxypropylmethylcellulose, 0.5% (w/v) of NaCl, 0.2% (w/v) of polyoxyl 35 castor oil (hereinafter also referred to as CO35), sodium hydroxide (appropriate amount), diluted hydrochloric acid (appropriate amount) and purified water (appropriate amount) was prepared.

### 2) Preparation of Ursodeoxycholic Acid Test Sample

To ursodeoxycholic acid was added Vehicle-C to give a 1% (w/v) solution (pH7.0). To ursodeoxycholic acid was added Vehicle-D to give a 1% (w/v) suspension (pH7.0).

### 6-2. Test Method

1) Each test sample (2.5 μL/eye) was instilled into both eyes of 8-month-old C57BL/6J mice with a Pipetman once per day (around 13:30) for 7 days.
2) Approximately 24 hours after the final instillation, the mice were euthanized by carbon dioxide inhalation, and then the eyeballs were extracted and rinsed with Hank's balanced salt solution (HBSS).
3) The sclera near the optic nerve was cut with a razor, the lens was removed through the incision, and the removed lens was immersed in HBSS.
4) The lens was placed on a glass slide, and an all-in-one fluorescence microscope BZ-9000 (Keyence) was used to capture an image of the lens (Image a).
5) Next, one cover glass (Corning^{(registered trade mark)} 22×22 mm Square) was placed on the lens, and an image in which the thickness of the lens changed due to the weight was similarly captured (Image b).
6) A change in the lens diameter was calculated from Equation 1 wherein the lens diameter of Image a is subtracted from the lens diameter of Image b, as described below. Then, the lens elasticity improvement of each sample group compared with the vehicle-D control group was calculated from Equation 2 described below. The mean of each group was based on 10 eyes.

Change in lens diameter=Lens diameter in Image b of each test sample−Lens diameter in Image a of each test sample (Equation 1)

Lens elasticity improvement of each sample group=Mean change in lens diameter of each Test sample group−Mean change in lens diameter of Vehicle-D control group (Equation 2)

### 6-3. Test Results and Discussion

The results are shown in Table 8.

TABLE 8

| | Lens elasticity |
|---|---|
| | improvement (μm) |
| 1% UDCA aqueous soluble starch | 14.9 |
| conversion product solution (QD) | |
| 1% UDCA suspension (QD) | 31.0 |

As shown in Table 8, the suspension pharmaceutical composition comprising UDCA showed a stronger lens elasticity improvement compared to the solution comprising an aqueous soluble starch conversion product and UDCA.

### INDUSTRIAL APPLICABILITY

The pharmaceutical composition of the present disclosure has an excellent tissue penetration of ursodeoxycholic acid or a salt thereof and is useful as a medicament.

1. A pharmaceutical composition comprising ursodeoxycholic acid or a salt thereof, a preservative, and water.

2. The pharmaceutical composition according to claim 1, wherein the preservative is selected from the group consisting of benzalkonium halide, boric acid or a salt thereof, and combinations thereof.

3. The pharmaceutical composition according to claim 1, wherein the preservative comprises benzalkonium halide, and optionally comprises boric acid or a salt thereof.

4. The pharmaceutical composition according to claim 1, wherein the preservative comprises benzalkonium halide and boric acid or a salt thereof.

5. The pharmaceutical composition according to claim 2, wherein the benzalkonium halide is selected from the group consisting of benzalkonium chloride, benzalkonium bromide, and a combination thereof.

6. The pharmaceutical composition according to claim 2, wherein the benzalkonium halide is benzalkonium chloride.

7. The pharmaceutical composition according to claim 2, wherein the boric acid or a salt thereof is selected from the group consisting of boric acid, borax, and a combination thereof.

8. The pharmaceutical composition according to claim 1, wherein the pharmaceutical composition has a pH greater than or equal to 8.0.

9. The pharmaceutical composition according to claim 1, wherein the pharmaceutical composition has a pH of 8.3 to 9.3.

10. The pharmaceutical composition according to claim 1, further comprising a nonionic surfactant.

11. The pharmaceutical composition according to claim 10, wherein the nonionic surfactant is polyoxyethylene sorbitan fatty acid ester.

12. The pharmaceutical composition according to claim 11, wherein the polyoxyethylene sorbitan fatty acid ester is polysorbate 80.

13. The pharmaceutical composition according to claim 1, further comprising a buffer.

14. The pharmaceutical composition according to claim 13, wherein the buffer is selected from the group consisting of phosphoric acid or a salt thereof, citric acid or a salt thereof, acetic acid or a salt thereof, carbonic acid or a salt thereof, tartaric acid or a salt thereof, ε-aminocaproic acid or a salt thereof, trometamol or a salt thereof, and combinations thereof.

15. The pharmaceutical composition according to claim 13, wherein the buffer is trometamol or a salt thereof.

16. The pharmaceutical composition according to claim 1, wherein the content of the ursodeoxycholic acid or a salt thereof in the pharmaceutical composition is 0.00001 to 5% (w/v).

17. The pharmaceutical composition according to claim 1, wherein the content of the ursodeoxycholic acid or a salt thereof in the pharmaceutical composition is 0.0003 to 0.9% (w/v).

18. The pharmaceutical composition according to claim 1, wherein the content of the preservative in the pharmaceutical composition is 0.0001 to 3.5% (w/v).

19. The pharmaceutical composition according to claim 2, wherein the content of the benzalkonium halide in the pharmaceutical composition is 0.0001 to 0.05% (w/v).

20. The pharmaceutical composition according to claim 2, wherein the content of the boric acid or a salt thereof in the pharmaceutical composition is 0.001 to 3% (w/v).

21. The pharmaceutical composition according to claim 10, wherein the content of the nonionic surfactant in the pharmaceutical composition is 0.001 to 30 parts by mass relative to 1 part by mass of ursodeoxycholic acid or a salt thereof.

22. The pharmaceutical composition according to claim 10, wherein the content of the nonionic surfactant in the pharmaceutical composition is 0.001 to 0.3% (w/v).

23. The pharmaceutical composition according to claim 10, wherein the content of the nonionic surfactant in the pharmaceutical composition is 0.03 to 0.09% (w/v).

24. The pharmaceutical composition according to claim 13, wherein the content of the buffer in the pharmaceutical composition is 0.001 to 5% (w/v).

25. The pharmaceutical composition according to claim 14, wherein the content of the trometamol or a salt thereof in the pharmaceutical composition is 0.001 to 2% (w/v).

26. The pharmaceutical composition according to claim 14, wherein the content of the trometamol or a salt thereof in the pharmaceutical composition is 0.05 to 0.9% (w/v).

27. The pharmaceutical composition according to claim 1, further comprising glycerin.

28. The pharmaceutical composition according to claim 1, which is a solution.

29. The pharmaceutical composition according to claim 1, wherein the pharmaceutical composition is administered into eye.

30. The pharmaceutical composition according to claim 1, wherein the pharmaceutical composition is an eye drop.

31. The pharmaceutical composition according to claim 1, wherein the pharmaceutical composition is for treating and/or preventing presbyopia, an eye disease accompanied by a decrease in lens elasticity, or an eye disease accompanied by a decrease in accommodative function of the eye.

32. (canceled)

33. A method for treating and/or preventing presbyopia, an eye disease accompanied by a decrease in lens elasticity, or an eye disease accompanied by a decrease in accommodative function of the eye, comprising administering to a subject in need thereof a therapeutically and/or prophylactically effective amount of the pharmaceutical composition according to claim 1.

34. A method for improving tissue penetration of ursodeoxycholic acid or a salt thereof in a pharmaceutical composition comprising ursodeoxycholic acid or a salt thereof and water, comprising adding a preservative.

35. A method for improving solution stability of the pharmaceutical composition according to claim 1, comprising adding a nonionic surfactant.

36. A method for suppressing an appearance change of a pharmaceutical composition comprising a nonionic surfactant and water, comprising adding ursodeoxycholic acid or a salt thereof.

37. A method for suppressing an appearance change of a pharmaceutical composition comprising a nonionic surfactant, a preservative, and water, comprising adding ursodeoxycholic acid or a salt thereof.

38. The method according to claim 35, wherein the preservative comprises benzalkonium halide.

39. A method for improving preservative efficacy of the pharmaceutical composition according to claim 1, comprising adding a buffer.
